(19) United States
(12) Patent Application Publication
(10) Pub. No.: US 2023/0226184 A1
(43) Pub. Date: Jul. 20, 2023

(54) IMPROVEMENTS IN ULTRASOUND-MEDIATED THERAPY

(71) Applicant: Bracco Suisse SA, Cadempino (CH)

(72) Inventors: Victor JEANNOT, Annecy (FR); Ebeline CACQUEVEL-BIHEL, Saint-Julien-en-Genevois (FR)

(73) Assignee: Bracco Suisse SA, Cadempino (CH)

(21) Appl. No.: 18/010,068

(22) PCT Filed: Jun. 15, 2021
(86) PCT No.: PCT/EP2021/066114

§ 371 Date: Dec. 13, 2022

(30) Foreign Application Priority Data
Jun. 15, 2020 (EP) 20180060.4

(51) Int. Cl. A61K 41/00; A61K 31/4418; A61K 31/517; A61K 33/06; A61K 9/00

(52) U.S. Cl. CPC A61K 41/0047; A61K 31/4418; A61K 31/517; A61K 33/06; A61K 9/0009

(57) ABSTRACT

The present invention relates to the field of ultrasound-mediated therapeutic treatments in combination with gas-filled microvesicles. It relates in particular to enhancing the efficacy of a combined therapeutic treatment of gas-filled microvesicles and ultrasound, by reducing the vasospasm effect caused by said therapeutic treatment.

### TECHNICAL FIELD

The present invention generally relates to the field of ultrasound-mediated therapeutic treatments in combination with gas-filled microvesicles.

In particular, it relates to a vasospasm inhibitor for use in enhancing the efficacy of a combined therapeutic treatment of gas-filled microvesicles and ultrasound for therapy. Moreover, it relates to a method for enhancing the efficacy of a combined therapeutic treatment of gas-filled microvesicles and ultrasound for therapy.

### BACKGROUND OF THE INVENTION

Gas-filled microvesicles (MV) are acoustic resonators that, when submitted to an acoustic field, scatter the received acoustic energy through different processes such as (i) oscillation, also called cavitation, which confers acoustic and mechanical properties to the gas-filled microvesicles, and (ii) heating.

In the last decades, acoustic properties of gas-filled microvesicles have been exploited mainly for Contrast-Enhanced Ultrasound imaging (CEUS). However, mechanical and heating properties of gas-filled microvesicles are currently under evaluation for the application in ultrasound-mediated therapies, such as promoting/increasing delivery of medicaments (Ref. 1) or genes, helping disrupting blood clots (Ref. 2), opening blood-brain barrier (Ref. 3), immunomodulation (Ref. 4), neuromodulation (Ref. 5), radiosensitization (Ref. 6), MV enhanced thermal ablation (Ref. 7), hyperthermia (Ref. 8), or helping in non-thermal tissue ablation (Ref. 9).

Therapeutic ultrasounds (US) usually consist of high-intensity ultrasounds waves, characterized by high mechanical index and long pulse duration, in general not suitable for diagnostic imaging.

Despite of the increasing number of applications of gas-filled microvesicles in ultrasound-mediated therapy, the Applicant has now observed that these combined therapeutic treatments may nevertheless suffer from a reduced efficacy under certain insonation conditions.

The Applicant has now unexpectedly found that the use of vasospasm inhibitors (VI) in combination with the use of gas-filled microvesicles and ultrasound-mediated therapy can improve the overall efficacy of the treatment.

As a matter of fact, the Applicant has observed that a (substantial) reduced efficacy in such therapeutic treatments can be associated with a transient vasomotor response occurring in a subject upon ultrasounds/gas-filled microvesicles exposure. While such phenomenon, also known as “vasospasm”, has been described in the literature (see for instance Ref. 10), it has never been associated, to the best of Applicant's knowledge, to a reduced efficacy of therapeutic treatments based on the combined use of gas-filled microvesicles and ultrasound.

Advantageously, the solution proposed by the Applicant allows to substantially limit or avoid the above-mentioned reduction of therapeutic efficacy, whereas said efficacy is in particular enhanced with respect to a combined microvesicles/ultrasounds-mediated treatment performed in the absence of a vasospasm inhibitor. View from another side, the use of such vasospasm inhibitor allows maintaining an acceptable level of therapeutic efficacy of the combined microvesicles/ultrasounds-mediated treatment.

Use of a vasospasm inhibitor and gas-filled microvesicles in combination with ultrasounds is known in the diagnostic imaging field.

For instance, Ref. 11 deals with improved methods for diagnostic imaging, involving the administration to a patient of a contrast agent and a coronary vasodilator.

Ref 12 deals with a combined preparation comprising an injectable aqueous gas dispersion and a vasodilator drug, namely adenosine, to be used to generate enhanced images.

Ref 13 relates to gas encapsulated acoustically responsive stabilized microbubbles comprising an encapsulated bioactive gas for promoting localized vasodilation in patients in need thereof by releasing said encapsulated bioactive agent.

According to Applicant's knowledge, the use of vasospasm inhibitors for limiting or avoiding a reduction of the therapeutic efficacy of the combined use of ultrasounds and gas-filled microvesicles in therapeutic treatments has thus never been described.

### SUMMARY OF THE INVENTION

An aspect of the invention relates to a vasospasm inhibitor (VI) for use in enhancing the efficacy of a combined therapeutic treatment of gas-filled microvesicles and ultrasound for therapy.

In an embodiment, said vasospasm inhibitor is selected from the group consisting of dihydropyridine calcium blockers, a-blockers and nitrovasodilators.

In a further embodiment, said vasospasm inhibitor is preferably selected from the group consisting of nimodipine, nifedipine, magnesium, prazosin and nitroglycerin; still more preferably said vasospasm inhibitor is nimodipine.

Said vasospasm inhibitor can be administered simultaneously or sequentially with respect to the suspension of gas filled microvesicles.

When the VI is administered simultaneously with the suspension of gas-filled microvesicles, it can be co-administered as a suspension of microvesicles comprising the VI or as two separate solutions which are co-administered.

When the VI is administered sequentially with respect to the suspension of gas-filled microvesicles, it is preferably administered in advance, e.g. from 1 second to 15 minutes before the suspension of gas-filled microvesicles, preferably from 5 seconds to 12 minutes before, more preferably at least 10 minutes before.

Another aspect of the present invention relates to a method of combined therapeutic treatment of gas-filled microvesicles and ultrasound for therapy, said method comprising the steps of:

a) administering a vasospasm inhibitor into the vascular system of a subject;

b) administering a suspension of gas-filled microvesicles into the vascular system of the subject;

c) applying ultrasounds for therapy to a region of interest of the subject.

In particular, the use of the vasospasm inhibitor in said method allows enhancing the efficacy of said combined therapeutic treatment.

In an embodiment, the drug delivery protocol of the invention relates to a method of combined therapeutic treatment of gas-filled microvesicles and ultrasound for therapy, said method comprising the steps of:

a′) administering a bioactive agent into the vascular system of a subject;

a) administering a vasospasm inhibitor into the vascular system of a subject;

b) administering a suspension of gas-filled microvesicles, into the vascular system of the subject;

c) applying ultrasounds for therapy to a region of interest of the subject.

In particular, the use of the vasospasm inhibitor in said method allows enhancing the efficacy of said combined therapeutic treatment.

In an embodiment of the present invention, step a′) and step a) of the disclosed method are carried out simultaneously or sequentially.

### FIGURES

FIG. 1: quantification of tumoral perfusion during treatment with US+VI. In upper panel: without VI pre-treatment; in lower panel: VI pre-treatment.

### DETAILED DESCRIPTION OF THE INVENTION

The Applicant has found that the administration of a vasospasm inhibitor (VI) with gas-filled microvesicles in ultrasound therapy can improve the efficacy of the therapeutic treatment.

As used herein, the term “efficacy” refers to the ability of a combination of therapeutic ultrasounds and gas-filled microvesicles (US-MV) to provide a beneficial therapeutic effect.

For instance, the efficacy of a combined use of US-MV for drug delivery can be estimated by its ability in enhancing the concentration of a bioactive molecule (e.g. therapeutic or contrast agents) in the vascular compartment of the target area; its degree of extravasation through blood vessels and/or its degree of intracellular delivery.

In the combined use of US-MV for sonothrombolysis (i.e. disruption of blood clots), the efficacy of the treatment can be estimated, for instance, by its ability in enhancing perfusion or inducing recanalization of blood vessel either in a direct way, e.g. by imaging follow-up (e.g. by angiography, Computed Tomography, laser doppler, contrast enhanced ultrasound imaging or Magnetic resonance imaging) or in an indirect way, e.g. by biological analysis of blood samples; electrocardiogram changes; and/or by assessment of clinical improvement(s) (e.g. neurological function or cardiac function).

The efficacy of a combined use of US-MV for MV enhanced thermal ablation can be assessed, for instance, by measuring the temperature increase of a specific volume of tissue and by the reduction of sonication time required to reach this temperature; by the reduction of acoustic energy or power required to reach thermal increase or by the follow-up of tissue damages by imaging.

The efficacy of a combined use of US-MV for MV enhanced hyperthermia can be assessed, for instance by measuring an increase of temperature in a specific volume of tissue (e.g by magnetic resonance thermometry, or thermocouple); by the reduction of acoustic energy or power required to achieve hyperthermia; by the increase of blood flow in the treated area (e.g. by angiography, Magnetic resonance imaging, Computed Tomography, laser doppler, contrast enhanced ultrasound imaging) by the follow up of drugs delivery and/or by similar estimation described below for “drug delivery”.

The efficacy of a combined use of US-MV for BBB opening can be evaluated, for instance, by its ability in enhancing the extravasation from vessels into the brain of a bioactive molecule (e.g therapeutic or contrast agents) through blood vessels.

Neuromodulation, i.e. the modulation of neuronal function induced by US+MV, is another therapeutic application field where the combined use of US-MV is applied. In this case, the efficacy of the combined US-MV treatment can be evaluated, for instance, by the variation of electrical activity of neurons.

The efficacy of a combined use of US-MV for application in radiosensitization, i.e the treatment of tumors by enhancing the effect of radiation therapy (RT) using US and MV, can be evaluated, for instance, by detecting the presence of treatment induced lesion as highlighted by any of the following: changes in tissue hemodynamics; an increase level of cell death and apoptosis in tissue exposed to US+RT; a reduction of tumor growth and/or an increased survival over the weeks.

The efficacy of a combined use of US-MV for application in Non-Thermal Ablation can be assessed, for instance, by detecting the presence of treatment induced lesion(s) as highlighted e.g. by a reduction in blood flow and/or by the follow-up necrosis and apoptosis in the sonicated tissue.

The combined use of US-MV can also be applied in anti-tumour therapy, exerting an anti-tumor effect by immunomodulation, i.e. the stimulation of an anti-tumor immune response. Since US-MV induced immunomodulation can be achieved through different mechanisms, such as thermal ablation, mild hyperthermia, mechanical destruction, or vessel permeabilization for delivery of immunotherapy drugs (e.g. DNA, mRNA, drugs or antibodies), it is expected that improving these mechanisms may improve the immunomodulation effects. The efficacy of the combined use of US-MV for application in immunomodulation can be assessed e.g by the induction or the modification of immune response; the modification of response to immunotherapy and/or the modification of a biological response (e.g. tumor growth).

In the present description and claims, the expression “combined use of ultrasound for therapy and a suspension of gas-filled microvesicles” (in short “combined use of US-MV) indicates any therapeutic treatment based on the use of therapeutic ultrasound waves in combination with a suspension of gas-filled microvesicles.

As mentioned above, these therapeutic treatments may be used for instance for increasing vessels permeabilization, e.g. for promoting/increasing delivery of medicaments or genes, for helping disrupting blood clots, opening blood brain barrier, immunomodulation, neuromodulation, radiosensitization, or also for helping Hyperthermia, MV enhanced thermal ablation and in non-thermal tissue ablation.

The expression “ultrasounds for therapy” as used herein indicates the use of ultrasounds to achieve biological effects in order to treat a disease or a disorder in a subject. In the present description and claims the expressions “US for therapy” or “therapeutic US” is used interchangeably.

Ultrasounds for therapy can be generated by different types of transducers. Suitable examples of transducers for ultrasounds for therapy are focused transducers or unfocused transducer, equipped by a single or multiple piezo-electrical element.

Usually ultrasounds for therapy are provided by dedicated platforms (e.g. coupled with MRI scanner) and clinical echograph platforms.

In order to achieve the biological effects, several parameters of the ultrasounds for therapy have to be considered.

The ultrasounds for therapy are generally characterized by a treatment duration preferably comprised between few seconds, i.e. 1 second, and 170 min. The expression “treatment duration” as used herein indicates the overall time span between the onset of the ultrasound application in the region of interest and its end. The unit of the treatment duration is time.

The pulse length characterizing the ultrasounds for therapy is generally comprised between 5 μs and 60 s, preferably comprised between 5 μs and 10 s, still more preferably the pulse length is comprised between 100 μs and 5 ms. The expression “pulse length” refers to the time from the start of a pulse (“on”) to the end of that pulse (“off”), indicating the actual time that the pulse is “on”. The unit of the pulse length is time (e.g microseconds to seconds).

The frequency of ultrasounds for therapy is generally comprised from 20 kHz up to 70 MHz, preferably from 0.10 MHz to 50 MHz, still more preferably from about 0.15 to about 2 MHz. The term “frequency” indicates the number of certain events that occur in a particular time duration. The unit of the frequency is Hertz (i.e. Vs).

The acoustic pressure of ultrasounds for therapy is generally comprised between 10 kPa and 100 MPa, preferably comprised from 20 kPa and 50 MPa, still more preferably comprised between 50 kPa and 25 MPa.

The acoustic intensity (watt/cm² or W/cm²) of ultrasounds for therapy is generally comprised between 0.1 and 990 W/cm², preferably comprised from 1.3 W/cm² and 21.5 W/cm², still more preferably comprised between 1.3 and 5 W/cm². The expression “acoustic intensity” refers to power carried by sound waves per unit area in a sound beam. The unit of the acoustic intensity is watts/cm².

Therapeutic ultrasound is in most cases carried out at different frequencies than diagnostic ultrasound. Specifically, it is desirable to perform therapeutic ultrasounds at lower frequencies in order to achieve low attenuation, whereas higher frequencies are employed in diagnostic ultrasound to obtain better resolution (Ref. 14).

Ultrasounds for therapy can also be distinguished from diagnostic ultrasounds due to their capability to induce biological effects (with or without combination with MV).

For therapy, ultrasound can induce effects not only through heating, but also through nonthermal mechanisms including acoustic cavitation, radiation force, shear stress and acoustic streaming/microstreaming, shock waves or other undetermined nonthermal processes. Thermal effect and acoustic cavitation are the most significant effects, and their mechanisms of action and biological effects are known in the field. As used herein, US for therapy indicates ultrasounds capable of inducing biological effects on a subject undergoing a therapeutic ultrasound treatment, including, for instance, local temperature increase, vessel permeabilization, vessel rupture and/or shear stress.

Thermal effects are dependent on the temperature and the duration of the treatment. For instance, low temperature increase (e.g. >43° C., 1 hour) will sensitize the tissue to chemotherapy or radiotherapy, whereas higher temperature increase (e.g. 56° C., 1 sec) will provoke irreversible damages like thermal coagulation and cell death.

Acoustic cavitation and mechanical effect of US for therapy cause a significant degree of mechanical and thermal effects as well as chemical and optical effects leading to various biological effects, such as local temperature increase, vessel permeabilization, vessel rupture, cell death and shear stress.

In an embodiment, US for therapy indicates US capable of inducing vessel permeabilization.

Ultrasounds for therapy are extensively used for tissue thermal ablation in various type of pathologies like symptomatic uterine fibroids, cancer (e.g. breast and liver prostate cancer), pain and neurological disorders but also for sonothrombolysis and Histotripsy.

The use of MV in combination with ultrasound for therapy enhances the effects of many different therapeutic responses where acoustic cavitation is known to be involved. Indeed, when injected, MV act as cavitation nuclei, lowering the threshold of acoustic cavitation. Tuning the US conditions, it is possible to control the cavitation regimen of MV, moving from a stable regimen of oscillation (stable cavitation) to a violent collapse for higher energy. This regimen of cavitation can be selectively used to either improve thermal ablation (MV enhanced thermal ablation), to dissolve blood clots (Sonothrombolysis), to permeabilize blood vessels (Blood brain barrier opening) to achieve specific drug or gene delivery, for immunomodulation, neuromodulation, radiosensitization, helping hyperthermia and the non-thermal ablation of tissue.

The term “gas-filled microvesicles” as used herein includes any structure comprising bubbles of gas of micrometric or nanometric size surrounded by an envelope or layer (including film-form layers) of a stabilizing material. The term includes what is known in the art as gas-filled liposomes, microbubbles, microspheres, microballoons or microcapsules. The stabilizing material can be any material typically known in the art including, for instance, surfactants, lipids, sphingolipids, oligolipids, glycolipids, phospholipids, proteins, polypeptides, carbohydrates, and synthetic or natural polymeric materials.

The term “precursor” of a gas-filled microvesicle includes any composition which, upon reconstitution with an aqueous carrier in the presence of a gas, will produce a suspension of gas-filled microvesicles. Said compositions typically include any of the above-cited stabilizing materials in dry powdered form (e.g. freeze-dried or spray-dried) capable of forming gas-filled microvesicles upon shaking an aqueous suspension thereof in the presence of a gas.

The term “microbubbles” includes aqueous suspensions in which the bubbles of gas are bounded at the gas/liquid interface by a very thin envelope (film) involving a stabilizing amphiphilic material disposed at the gas to liquid interface. Microbubble suspensions can be prepared by contacting a suitable precursor thereof, such as powdered amphiphilic materials (e.g. freeze-dried preformed liposomes or freeze-dried or spray-dried phospholipid solutions) with air or other gas and then with an aqueous carrier, while agitating to generate a microbubble suspension which can then be administered, preferably shortly after its preparation.

Gas-filled microbubbles are generally stabilized by one or more amphiphilic component. Amphiphilic components suitable for forming a stabilizing envelope of microbubbles comprise, for instance, phospholipids; lysophospholipids; fatty acids, such as palmitic acid, stearic acid, arachidonic acid or oleic acid; lipids bearing polymers, such as chitin, hyaluronic acid, polyvinylpyrrolidone or polyethylene glycol (PEG), also referred as “pegylated lipids”; lipids bearing sulfonated mono- di-, oligo- or polysaccharides; cholesterol, cholesterol sulfate or cholesterol hemisuccinate; tocopherol hemisuccinate; lipids with ether or ester-linked fatty acids; polymerized lipids; diacetyl phosphate; dicetyl phosphate; ceramides; polyoxyethylene fatty acid esters (such as polyoxyethylene fatty acid stearates), polyoxyethylene fatty alcohols, polyoxyethylene fatty alcohol ethers, polyoxyethylated sorbitan fatty acid esters, glycerol polyethylene glycol ricinoleate, ethoxylated soybean sterols, ethoxylated castor oil or ethylene oxide (EO) and propylene oxide (PO) block copolymers; sterol aliphatic acid esters including, cholesterol butyrate, cholesterol iso-butyrate, cholesterol palmitate, cholesterol stearate, lanosterol acetate, ergosterol palmitate, or phytosterol n-butyrate; sterol esters of sugar acids including cholesterol glucuronides, lanosterol glucuronides, 7-dehydrocholesterol glucuronide, ergosterol glucuronide, cholesterol gluconate, lanosterol gluconate, or ergosterol gluconate; esters of sugar acids and alcohols including lauryl glucuronide, stearoyl glucuronide, myristoyl glucuronide, lauryl gluconate, myristoyl gluconate, or stearoyl gluconate; esters of sugars with aliphatic acids including sucrose laurate, fructose laurate, sucrose palmitate, sucrose stearate, glucuronic acid, gluconic acid or polyuronic acid; saponins including sarsasapogenin, smilagenin, hederagenin, oleanolic acid, or digitoxigenin; glycerol or glycerol esters including glycerol tripalmitate, glycerol distearate, glycerol tristearate, glycerol dimyristate, glycerol trimyristate, glycerol dilaurate, glycerol trilaurate, glycerol dipalmitate; N-succinyldioleylphosphatidylethanolamine; 1,2-dioleyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine or palmitoylhomocysteine; alkylamines or alkylammonium salts, comprising at least one (C₁₀-C₂₀), preferably (C₁₄-C₁₈), alkyl chain, such as, for instance, N-stearylamine, N,N′-distearylamine, N-hexadecylamine, N,N′-dihexadecylamine, N-stearylammonium chloride, N,N′-distearylammonium chloride, N-hexadecylammonium chloride, N,N′-dihexadecylammonium chloride, dimethyldioctadecylammonium bromide (DDAB), hexadecyltrimethylammonium bromide (CTAB); tertiary or quaternary ammonium salts comprising one or preferably two (C₁₀-C₂₀), preferably (C₁₄-C₁₈), acyl chain linked to the N-atom through a (C₃-C₆) alkylene bridge, such as, for instance, 1,2-distearoyl-3-trimethylammonium-propane (DSTAP), 1,2-dipalmitoyl-3-trimethylammonium-propane (DPTAP), 1,2-oleoyl-3-trimethylammonium-propane (DOTAP), 1,2-distearoyl-3-dimethylammonium-propane (DSDAP); and mixtures or combinations thereof.

As used herein, the term “phospholipid” is intended to encompass any amphiphilic phospholipid compound, the molecules of which are capable of forming a stabilizing film of material (typically in the form of a mono-molecular layer) at the gas-water boundary interface in the final microbubbles' suspension. Accordingly, these materials are also referred to in the art as “film-forming phospholipids”.

Phospholipids typically contain at least one phosphate group and at least one, preferably two, lipophilic long-chain hydrocarbon group.

Examples of suitable phospholipids include esters of glycerol with one or preferably two (equal or different) residues of fatty acids and with phosphoric acid, wherein the phosphoric acid residue is in turn bound to a hydrophilic group, such a, for instance, choline (phosphatidylcholines—PC), serine (phosphatidylserines—PS), glycerol (phosphatidyl-glycerols—PG), ethanolamine (phosphatidylethanolamines—PE), inositol (phosphatidylinositol—PI). Esters of phospholipids with only one residue of fatty acid are generally referred to in the art as the “lyso” forms of the phospholipid or “lysophospholipids”. Fatty acids residues present in the phospholipids are in general long chain aliphatic acids, typically containing from 12 to 24 carbon atoms, preferably from 14 to 22; the aliphatic chain may contain one or more unsaturations or is preferably completely saturated. Examples of suitable fatty acids included in the phospholipids are, for instance, lauric acid, myristic acid, palmitic acid, stearic acid, arachidic acid, behenic acid, oleic acid, linoleic acid, and linolenic acid. Preferably, saturated fatty acids such as myristic acid, palmitic acid, stearic acid and arachidic acid are employed.

Further examples of phospholipid are phosphatidic acids, i.e. the diesters of glycerol-phosphoric acid with fatty acids; sphingolipids such as sphingomyelins, i.e. those phosphatidylcholine analogs where the residue of glycerol diester with fatty acids is replaced by a ceramide chain; cardiolipins, i.e. the esters of 1,3-diphosphatidylglycerol with a fatty acid; glycolipids such as gangliosides GM1 (or GM2) or cerebrosides; glucolipids; sulfatides and glycosphingolipids.

As used herein, the term phospholipids include either naturally occurring, semisynthetic or synthetically prepared products that can be employed either singularly or as mixtures.

Examples of naturally occurring phospholipids are natural lecithins (phosphatidylcholine (PC) derivatives) such as, typically, soya bean or egg yolk lecithins.

Examples of semisynthetic phospholipids are the partially or fully hydrogenated derivatives of the naturally occurring lecithins. Preferred phospholipids are fatty acids di-esters of phosphatidylcholine, ethylphosphatidylcholine, phosphatidylglycerol, phosphatidic acid, phosphatidylethanolamine, phosphatidylserine, phosphatidylinositol or of sphingomyelin.

Specific examples of phospholipids are, for instance, dilauroyl-phosphatidylcholine (DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), 1,2 Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroyl-phosphatidylglycerol (DLPG) and its alkali metal salts, diarachidoylphosphatidyl-glycerol (DAPG) and its alkali metal salts, dimyristoylphosphatidylglycerol (DMPG) and its alkali metal salts, dipalmitoylphosphatidylglycerol (DPPG) and its alkali metal salts, distearoylphosphatidylglycerol (DSPG) and its alkali metal salts, dioleoyl-phosphatidylglycerol (DOPG) and its alkali metal salts, dimyristoyl phosphatidic acid (DMPA) and its alkali metal salts, dipalmitoyl phosphatidic acid (DPPA) and its alkali metal salts, distearoyl phosphatidic acid (DSPA), diarachidoylphosphatidic acid (DAPA) and its alkali metal salts, dimyristoyl-phosphatidylethanolamine (DMPE), dipalmitoylphosphatidylethanolamine (DPPE), distearoyl phosphatidyl-ethanolamine (DSPE), dioleylphosphatidyl-ethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distearoylphosphatidylinositol (DSPI), dioleoyl-phosphatidylinositol (DOPI). In particular phospholipids selected from DAPC, DSPC, DPPC, DMPA, DPPA, DSPA, DMPG, DPPG, DSPG, DMPS, DPPS, DSPS, Ethyl-DSPC, Ethyl-DPPC or mixtures thereof can be used, more in particular DPPG, DPPS and DSPC. Phospholipids may further include phospholipids modified by linking a hydrophilic polymer, such as polyethyleneglycol (PEG) or polypropyleneglycol (PPG), thereto. Preferred polymer-modified phospholipids include “pegylated phospholipids”, i.e. phospholipids bound to a PEG polymer. Examples of pegylated phospholipids are pegylated phosphatidylethanolamines (“PE-PEGs” in brief) i.e. phosphatidylethanolamines where the hydrophilic ethanolamine moiety is linked to a PEG molecule of variable molecular weight (e.g. from 300 to 20000 daltons, preferably from 500 to 5000 daltons), such as DPPE-PEG (or DSPE-PEG, DMPE-PEG, DAPE-PEG or DOPE-PEG). For example, DPPE-PEG2000 refers to DPPE having attached thereto a PEG polymer having a mean average molecular weight of about 2000. Examples of typical pegylated phospholipids include DPPE-PEG2000, DSPE-PEG2000, DPPE-PEG5000, DSPE-PEG5000.

Pegylated derivatives of phosphatidylethanolamines, in particular DPPE-PEG and/or DSPE-PEG, are typically used in admixture with any of the previously mentioned phospholipids.

Typically, the phospholipid is the main component of the stabilizing envelope of microbubbles, amounting to at least 50% (w/w) of the total amount of components forming the envelope of the gas filled microbubbles, preferably at least 75%. In some of the preferred embodiments, substantially the totality of the envelope (i.e. at least 90% w/w) can be formed of phospholipids.

The phospholipids can conveniently be used in admixture with any of the above listed amphiphilic compounds. Thus, for instance, lipids such as cholesterol, ergosterol, phytosterol, sitosterol, lanosterol, tocopherol, propyl gallate or ascorbyl palmitate, fatty acids such as myristic acid, palmitic acid, stearic acid, arachidic acid and derivatives thereof or butylated hydroxytoluene and/or other non-phospholipid compounds can optionally be added to one or more of the foregoing phospholipids, e.g. in proportions preferably ranging from zero to 50% by weight, more preferably up to 25%. Particularly preferred is palmitic acid.

Other excipients or additives may be present either in the dry formulation of the microbubbles or may be added together with the aqueous carrier used for the reconstitution thereof, without necessarily being involved (or only partially involved) in the formation of the stabilizing envelope of the microbubble. These include pH regulators, osmolality adjusters, viscosity enhancers, emulsifiers, bulking agents, etc. and may be used in conventional amounts. For instance, compounds like polyoxypropylene glycol and polyoxyethylene glycol as well as copolymers thereof can be used. Examples of viscosity enhancers or stabilizers are compounds selected from linear and cross-linked poly- and oligo-saccharides, sugars and hydrophilic polymers such as polyethylene glycol.

As the preparation of gas-filled microbubbles may involve a freeze drying or spray drying step, it may be advantageous to include in the formulation a lyophilization additive, such as an agent with cryoprotective and/or lyoprotective effect and/or a bulking agent, for example an amino-acid such as glycine or histidine; a carbohydrate, e.g. a sugar such as sucrose, mannitol, maltose, trehalose, glucose, lactose or a cyclodextrin, or a polysaccharide such as dextran, chitosan and its derivatives (for example: carboxymethyl chitosan, trimethyl chitosan); or a polyoxyalkyleneglycol such as polyethylene glycol.

Microbubbles can be produced according to any known method in the art. Typically, the manufacturing method involves the preparation of a dried powdered material comprising an amphiphilic material as indicated above, preferably by lyophilization (freeze drying) of an aqueous or organic suspension comprising said material. Examples of preparation of microbubbles and precursors thereof are disclosed for instance in Ref. 15 or Ref. 16. According to this latter manufacturing procedure, a phospholipid (and other optional film-forming materials), a lyoprotecting agent and other optional additives can be dispersed in a mixture of water and a water immiscible organic solvent under agitation, to form a microemulsion. The obtained microemulsion, which contains microdroplets of solvent surrounded and stabilized by the phospholipid (and optionally by other amphiphilic film-forming compounds and/or additives), is then lyophilized according to conventional techniques to obtain a lyophilized material, which is stored (e.g. in a vial in the presence of a suitable gas) and which can be reconstituted with an aqueous carrier to finally give a gas-filled microbubbles suspension.

Alternatively, calibrated gas-filled microbubbles can be prepared by using a flow-focusing device, as disclosed for instance in Ref. 17.

The freeze-dried product will generally be in the form of a powder or a cake, and can be stored (e.g. in a vial) in contact with the desired gas. The product is readily reconstitutable in a suitable physiologically acceptable aqueous liquid carrier, which is typically injectable, to form the gas-filled microbubbles, upon gentle agitation of the suspension. Suitable physiologically acceptable liquid carriers are sterile water, aqueous solutions such as saline (which may advantageously be balanced so that the final product for injection is not hypotonic), or solutions of one or more tonicity adjusting substances such as salts or sugars, sugar alcohols, glycols or other non-ionic polyol materials (eg. glucose, sucrose, sorbitol, mannitol, glycerol, polyethylene glycols, propylene glycols and the like), chitosan derivatives, such as carboxymethyl chitosan, trimethyl chitosan or gelifying compounds, such as carboxymethylcellulose, hydroxyethyl starch or dextran.

Other suitable gas-filled microvesicles are referred to in the art as “microballoons” “microcapsules” or “microspheres”. These gas-filled microvesicles include suspensions in which the bubbles of gas are surrounded by a solid material envelope which can be e.g. polymeric (natural or synthetic), proteinaceous, of a water insoluble lipid or of any combination thereof. Examples of microballoons made of polymeric materials are disclosed, for instance, in Ref. 18. Examples of microcapsules made from an insoluble lipid (e.g. tripalmitin or tristearin) are disclosed for instance in Ref. 19. Microspheres having a proteinaceous envelope (e.g. natural proteins such as albumin) are disclosed e.g. in Ref. 20.

Any biocompatible gas, gas precursor or mixture thereof may be employed to fill the above microvesicles (hereinafter also identified as “microvesicle-forming gas”).

The gas may comprise, for example, air; nitrogen; oxygen; carbon dioxide; hydrogen; nitrous oxide; a noble or inert gas such as helium, argon, xenon or krypton; a low molecular weight hydrocarbon (e.g. containing up to 7 carbon atoms), for example an alkane such as methane, ethane, propane, butane, isobutane, pentane or isopentane, a cycloalkane such as cyclobutane or cyclopentane, an alkene such as propene, butene or isobutene, or an alkyne such as acetylene; an ether; a ketone; an ester; halogenated gases, preferably fluorinated gases, such as or halogenated, fluorinated or perfluorinated low molecular weight hydrocarbons (e.g. containing up to 7 carbon atoms); or a mixture of any of the foregoing. Where a halogenated hydrocarbon is used, preferably at least some, more preferably all, of the halogen atoms in said compound are fluorine atoms.

Fluorinated gases are preferred, in particular perfluorinated gases. Fluorinated gases include materials which contain at least one fluorine atom such as, for instance fluorinated hydrocarbons (organic compounds containing one or more carbon atoms and fluorine); sulfur hexafluoride; fluorinated, preferably perfluorinated, ketones such as perfluoroacetone; and fluorinated, preferably perfluorinated, ethers such as perfluorodiethyl ether. Preferred compounds are perfluorinated gases, such as SF₆ or perfluorocarbons (perfluorinated hydrocarbons), i.e. hydrocarbons where all the hydrogen atoms are replaced by fluorine atoms, which are known to form particularly stable microbubble suspensions. The term perfluorocarbon includes saturated, unsaturated, and cyclic perfluorocarbons. Examples of biocompatible, physiologically acceptable perfluorocarbons are: perfluoroalkenes, such as perfluoromethane, perfluoroethane, perfluoropropanes, perfluorobutanes (e.g. perfluoro-n-butane, optionally in admixture with other isomers such as perfluoro-isobutane), perfluoropentanes, perfluorohexanes or perfluoroheptanes; perfluoroalkenes, such as perfluoropropene, perfluorobutenes (e.g. perfluorobut-2ene) or perfluorobutadiene; perfluoroalkynes (e.g. perfluorobut-2-yne); and perfluorocycloalkanes (e.g. perfluorocyclobutane, perfluoromethylcyclobutane, perfluorodimethylcyclobutanes, perfluorotrimethylcyclobutanes, perfluorocyclopentane, perfluoromethylcyclopentane, perfluorodimethylcyclopentanes, perfluorocyclohexane, perfluoromethylcyclohexane and perfluorocycloheptane). Preferred saturated perfluorocarbons include, for example, CF₄, C₂F₆, C₃F₈, C₄F₈, C₄F₈, C₅F₁₂ and C₆F₁₂.

It may also be advantageous to use a mixture of any of the above gases in any ratio. For instance, the mixture may comprise a conventional gas, such as nitrogen, air or carbon dioxide and a gas forming a stable microbubble suspension, such as sulfur hexafluoride or a perfluorocarbon as indicated above. Examples of suitable gas mixtures can be found, for instance, in Ref. 21, which is herein incorporated by reference. The following combinations are particularly preferred: a mixture of gases (A) and (B) in which the gas (B) is a fluorinated gas, selected among those previously illustrated, including mixtures thereof, and (A) is selected from air, oxygen, nitrogen, carbon dioxide or mixtures thereof. The amount of gas (B) can represent from about 0.5% to about 95% v/v of the total mixture, preferably from about 5% to 80%.

Particularly preferred gases are SF₆, C₃F₈, C₄F₁₀ or mixtures thereof, optionally in admixture with air, oxygen, nitrogen, carbon dioxide or mixtures thereof.

In certain circumstances it may be desirable to include a precursor to a gaseous substance (i.e. a material that is capable of being converted to a gas in vivo). Preferably the gaseous precursor and the gas derived therefrom are physiologically acceptable. The gaseous precursor may be pH-activated, photo-activated, temperature activated, etc. For example, certain perfluorocarbons may be used as temperature activated gaseous precursors. These perfluorocarbons, such as perfluoropentane or perfluorohexane, have a liquid/gas phase transition temperature above room temperature (or the temperature at which the agents are produced and/or stored) but below body temperature; thus, they undergo a liquid/gas phase transition and are converted to a gas within the human body.

While commercially available gas-filled microvesicles are approved for diagnostic use only, such as SonoVue®/Lumason (Bracco) Definity/Luminity (Lantheus) or Optison (GE Healthcare), their off-label use in therapeutic application is described in the art (see for example Ref. 22 and Ref. 23).

The Applicant has now observed that the therapeutic method based on the combined use of ultrasounds for therapy and gas-filled microvesicles (US-MV) may suffer from reduced efficacy.

Although not willing to be bound to any particular theory, the Applicant has observed that such reduced efficacy can be associated to a certain extent to a transient vasomotor response occurring in the subject, and particularly in the region under treatment, after US/gas-filled microvesicles exposure, known as “vasospasm”.

Vasospasm

In the present description and claims, the expression “vasospasm” indicates a strong vasomotor response induced by the combined use of ultrasound for therapy and a suspension of gas-filled microvesicles.

The generation and effects of this (transient) vasomotor response are described for instance in Ref. 10.

Although not willing to be bound to any particular theory, the reduced efficacy of the combined treatment of gas-filled microvesicles and ultrasound-mediated therapy can be associated to a certain extent to this transient vasomotor response occurring in the subject, and particularly in the region under treatment, after US/gas-filled microvesicles exposure.

The Applicant has now unexpectedly found that the overall efficacy of the combined treatment of gas-filled microvesicles and ultrasound-mediated therapy can be improved by the use of vasospasm inhibitors (VI).

The vasospasm induced by the combination of gas-filled microvesicles and ultrasounds for therapy mainly endows to a transient hypoperfusion, i.e. a reduction of the blood flow deriving from the vasoconstriction. In fact, the blood flow reduction resulting from the vessel constriction may as well affect the continuous transport of gas-filled microvesicles through the acoustic field, leading to a shortage of gas-filled microvesicles in the focal region and substantially reducing the effects of the ultrasounds pulse or make them substantially ineffective.

Advantageously, the solution proposed by the Applicant allows to substantially limit or avoid the above-mentioned reduction of therapeutic efficacy, whereas said efficacy is in particular enhanced with respect to a combined microvesicles/ultrasounds-mediated treatment performed in the absence of a vasospasm inhibitor. View from another side, the use of such vasospasm inhibitor allows maintaining an acceptable level of therapeutic efficacy of the combined microvesicles/ultrasounds-mediated treatment.

Vasospasm Inhibitor

As used herein, the expression “vasospasm inhibitor” (VI) refers to a substance able to enhance the overall efficacy of the combined treatment of gas-filled microvesicles and ultrasound for therapy, in particular by substantially reducing or avoiding the occurrence of vasospasm of blood vessels caused by the US-MV treatment.

A vasospasm inhibitor can, for instance, induce relaxation of the vascular smooth muscle cells through different mechanisms mainly based on lowering intracellular calcium concentration and inducing dephosphorylation (really substitution of ATP for ADP) of myosin.

Among all the compounds known to act as vasospasm inhibitors, the Applicant has surprisingly found that the vasospasm inhibitors grouped into the classes of dihydropyridine calcium channel blockers, a-adrenoceptor antagonists and nitrovasodilators are in particular able to enhance the overall efficacy of the combined treatment of gas-filled microvesicles and ultrasound for therapy.

Vasospasm inhibitors classified as calcium blockers (or calcium antagonists or calcium channel blockers) inhibit the calcium-induced contractions of vascular smooth muscle, by blocking the calcium ion influx into the vascular smooth muscle cells.

Preferred calcium blockers VI can be dihydropyridine calcium blockers.

Examples of dihydropyridine calcium blockers include amlodipine, aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine and pranidipine, particularly preferred being nifedipidine and nimodipine.

Preferably the VI is nifedipine or nimodipine, more preferably nimodipine.

Magnesium, which has similar properties as calcium antagonist and is considered a physiological calcium blocker can also be used as VI.

Vasospasm inhibitors classified as a-adrenoceptor antagonists (or a-blockers) lock the effect of alpha-1-adrenergic receptors in the vascular smooth muscle causing vasodilation. Examples of a-adrenoceptor antagonists include alfuzosin, doxazosin, prazosin silodosin, tamsulosin and terazosin, particularly preferred being prazosin.

Vasospasm inhibitors can be also classified as nitrovasodilators. The term “nitrovasodilators” indicates pharmacologic sources of nitric oxide, usually as organic nitrate vasodilators that can be metabolically converted to biologically activated nitric oxide. Suitable examples of nitrovasodilators usually administered exogenously are diethylene glycol dinitrate, glyceryl trinitrate (nitroglycerin), isosorbide mononitrate and dinitrate, itramin tosilate, pentaerithrityl tetranitrate, propatylnitrate, sinitrodil, tenitramine and trolnitrate, particularly preferred being nitroglycerin.

Nitric oxide leads to relaxation of vascular smooth muscle by stimulation of soluble guanylate cyclase, causing increased intracellular levels of cyclic guanosine monophosphate (cGMP).

In an embodiment, said vasospasm inhibitor is selected from the group consisting of dihydropyridine calcium blockers, a-blockers and nitrovasodilators.

In a further embodiment of this invention, said vasospasm inhibitor is preferably selected from the group consisting of nimodipine, nifedipine, magnesium, prazosin and nitroglycerin; still more preferably said vasospasm inhibitor is nimodipine.

In general the vasospasm inhibitor is substantially unbound to the gas-filled microvesicles.

The term “unbound” indicates that the vasospasm inhibitor has substantially no physical or chemical interaction with the gas-filled microvesicles; more in particular, the VI is not bound to the gas-filled microvesicles either through a covalent bond or via non-covalent binding (e.g. physical and/or electrostatic interactions).

In an embodiment, the VI is comprised in a separate pharmaceutical composition, not physically connected to the gas filled microvesicles suspension.

Examples of separate pharmaceutical composition comprising the vasospasm inhibitor include commercial formulations available on the market, such as oral dosage forms and parenteral dosage forms for both traditional and/or controlled-release.

The vasospasm inhibitor can be administered simultaneously or sequentially with the suspension of gas filled microvesicles.

The expression “simultaneous administration” as used herein indicates the administration of the VI and the gas-filled microvesicles at the same time or at substantially the same time, the administration route being identical or different.

According to this invention, the expression “sequential administration” indicates the administration of the VI and the gas-filled microvesicles at different times, the administration route being identical or different.

In an embodiment of the invention, the VI is administered simultaneously with the suspension of gas-filled microvesicles.

For instance, said VI can be added directly to the suspension of freeze-dried gas-filled microvesicles immediately before their administration. Alternatively, the VI can be added to the physiologically acceptable aqueous carrier (e.g. saline) that is used for the reconstitution of the freeze-dried gas-filled microvesicles.

In an embodiment of this invention, said vasospasm inhibitor is magnesium.

In a further embodiment, magnesium is administered simultaneously with respect to the administration of the suspension of gas filled microvesicles by diluting such microvesicles in magnesium solution.

In an alternative embodiment of the invention, the VI is administered sequentially to the suspension of gas-filled microvesicles.

In a preferred embodiment, the VI is administered from 1 second to 15 minutes before the suspension of gas-filled microvesicles, preferably from 5 seconds to 12 minutes before, still more preferably at least 10 minutes before.

The suspension of gas-filled microvesicles and the VI can be administered simultaneously or sequentially by intravenous injection. Alternatively, the suspension of gas-filled microvesicles can be administered by intravenous injection while the VI is administered orally, either simultaneously or sequentially.

The VI is preferably administered by intravenous injection at the typical approved therapeutic dose.

The expression “approved therapeutic dose” indicates the dose of a vasospasm inhibitor which has been approved by a regulatory agency (e.g. FDA and EMA) for their use. For instance, the approved therapeutic use can be expressed by VI dose per weight of subject to be treated (mg/kg), or by VI dose per day (mg/day), or VI weight per time unit (mg/hour).

According to the present invention, the suspension of gas-filled microvesicles can be administered either with a continuous infusion of the suspension or by injecting at least one bolus of a certain volume of the suspension to the subject.

For example, the total amount of microvesicles for each injected volume of suspension can vary from 6×10⁵ to 15×10⁹ microvesicles per kg of patient, preferably from 1×10⁷ to 12×10⁹ microvesicles per kg, even more preferably from 2×10⁷ to 10×10⁹ microvesicles per kg. The volume of each injected bolus is adapted to the required total amount of microvesicles to be injected (which generally depends on the specific treatment to be performed), taking into account the concentration of microvesicles in the injected suspension and the weight of the patient. As a general rule, for concentrations of microvesicles in the suspension of from about 1×10⁸ to about 3×10⁹ microvesicles/mL, the bolus volume may vary from 0.01 to 120 mL, preferably from 0.2 to 60 mL and even more preferably from 0.4 to 20 mL.

In the present description and claims, the expression “infusion administration” indicates an administration procedure which may be performed by any suitable continuous administration means, including, for instance, by gravity (e.g. saline infusion bag) or with a dedicated device, e.g. a controlled volume release (rotating) syringe.

The expression “bolus administration” refers to a single intravascular injection of a certain volume of a suspension of gas-filled microvesicles e.g. by means of a syringe and a catheter placed in a peripheral vein. The administration of a bolus is generally performed within few seconds (e.g. from 5 to 120 seconds, preferably not more than 60 seconds).

In an embodiment of the invention, the administration of said suspension of gas-filled microvesicles is administered as at least one bolus to the patient, preferably as at least one bolus, and up to 4 boluses, e.g. every 5 minutes.

In a further embodiment of the invention, the administration of said suspension of gas-filled microvesicles comprises administering an effective amount of gas filled microvesicles.

To this extent, and unless otherwise provided, the term “effective dose” or “effective amount”, as used herein, refers to any amount of a suspension of gas-filled microvesicles according to the invention that is sufficient to fulfil its intended therapeutic purpose(s): e.g., for example, to improve the drug delivery of a therapeutic agent in the region of interest.

The application of the therapeutic ultrasound on the region of interest can occur at any time before, during or after the administration of the gas-filled microvesicles. In general, it is preferable to start insonation after the administration of the VI. In certain embodiments, when VI and microvesicles are administered sequentially (e.g. first VI and then MV), insonation of the region of interest can be started at or immediately after the administration of the microvesicles. Alternatively, when VI and MV are administered simultaneously, insonation of the region of interest can be started at or immediately after the simultaneous administration. In certain embodiments, the arrival of the microvesicles in the region of interest can be determined (e.g. through physiological parameter or through contrast echographic imaging of the region of interest) and the insonation can be started when a desired amount of microvesicles have reached the region of interest.

The expression “region of interest” as used herein indicates a body part, organ or tissue of a subject which undergoes to a therapeutic treatment according to the invention.

According to present description and claims, the terms “subject” or “patient” refer to a living human or animal patient, and, preferably a human being undergoing the ultrasound-mediated therapy of this invention.

An aspect of the present invention relates to a method for enhancing the efficacy of a combined therapeutic treatment of gas-filled microvesicles and ultrasound for therapy, said method comprising the steps of:

a) administering a vasospasm inhibitor into the vascular system of a subject;

b) administering a suspension of gas-filled microvesicles into the vascular system of the subject;

c) applying ultrasounds for therapy to a region of interest of the subject.

Said vasospasm inhibitor is preferably selected from the group consisting of dihydropiridine calcium blockers, a-blockers and nitrovasodilators; more preferably it is selected from the group consisting of nimodipine, nifedipine, magnesium, prazosin and nitroglycerin; still more preferably said vasospasm inhibitor is nimodipine.

The vasospasm inhibitor is preferably administered, for instance by intravenous injection into the vascular system of a subject, at the approved therapeutic dosage.

The suspension of gas-filled microvesicles can be administered either with a continuous infusion or by injecting at least one bolus of a certain volume of the suspension to the subject; more preferably said suspension of gas-filled microvesicles is administered as at least one bolus to the subject.

The suspension of gas filled microvesicles is administered at an effective dose to the subject.

In an embodiment of the present invention, step a) and step b) of the disclosed method are carried out simultaneously or sequentially.

In a preferred embodiment, step a) is carried out simultaneously with step b).

In an alternative embodiment, step a) is carried out sequentially to step b). Preferably, step a) is carried out from 1 second to 15 minutes before the step b), more preferably from 5 seconds to 12 minutes before, still more preferably at least 10 minutes before.

In a further embodiment, step b) is carried out sequentially to step a).

Acoustic parameters, such as acoustic pressure, acoustic intensity and pulse length, are those described above.

In particular, at step c) of the method of the present invention, the ultrasounds for therapy have an acoustic pressure comprised between 100 kPa and 900 kPa, preferably comprised between 200 kPa and 800 kPa.

In a further embodiment, at step c) of the method of the present invention, the ultrasounds for therapy have an acoustic intensity comprised between 0.1 and 990 W/cm², preferably comprised between 1.3 W/cm² and 21.5 W/cm².

In a still further embodiment of this invention, at step c) said ultrasounds for therapy have a pulse length comprised between 5 μs and 60 s, preferably comprised between 5 μs 10 s, still more preferably the pulse length is 1 ms.

In a still further embodiment, at step c) said ultrasounds for therapy are applied for a time comprised between 1 s and 170 minutes, preferably between 2 and 10 minutes.

In a still further embodiment, at step c) said ultrasounds for therapy have a frequency comprised between 20 kHz and 70 MHz.

According to an embodiment, step c) is carried out after step b) (e.g. within seconds from administration of the microvesicles).

Alternatively, step c) can be carried out between step a) and step b). For instance, the sonication can be started after the administration of the VI (e.g. 1 to 15 minutes), before or simultaneously with the injection of the gas filled microvesicles.

In a general embodiment, a vasospasm inhibitor is used in a combined therapeutic treatment of gas-filled microvesicles and ultrasounds (for enhancing its efficacy), wherein said combined therapeutic treatment induces vessel permeabilization, e.g. for allowing or enhancing extravasation of a bioactive agent. In a specific embodiment, said combined therapeutic treatment induces a blood-brain barrier opening or disruption (BBB opening/disruption), e.g. combined with the delivery of a bioactive agent. In another specific embodiment said combined therapeutic treatment induces tumour perfusion, e.g. for the delivery of a bioactive agent.

In another embodiment a vasospasm inhibitor is used in a combined therapeutic treatment of gas-filled microvesicles and ultrasounds (for enhancing its efficacy) for dissolving blood clots (sonothrombolysis).

In a further embodiment, a vasospasm inhibitor is used in a combined therapeutic treatment of gas-filled microvesicles and ultrasounds for improving thermal ablation (MV enhanced thermal ablation).

In still further embodiments, the vasospasm inhibitor can be used for enhancing the efficacy of a combined therapeutic treatment of gas-filled microvesicles and ultrasounds for immunomodulation, neuromodulation, radiosensitization, helping hyperthermia or for non-thermal ablation of tissues.

As demonstrated by the Applicant, the administration of a VI is able to enhance the drug delivery efficacy of the combined therapeutic treatment US-MV. In certain embodiments, the use of the VI allowed to increase extravasation of a bioactive agent in the surrounding tumour tissue of almost two times compared with the extravasation obtained with only the combined US-MV treatment without VI.

In the present invention the expression “drug delivery” indicates a therapeutic protocol comprising the administration of at least a bioactive agent, said bioactive agent being different from the vasospasm inhibitor.

Bioactive agent includes any molecule, compound, formulation or material capable of being used in the ultrasound-mediated therapeutic treatments in combination with gas-filled microvesicles, which are capable of producing a biologically or therapeutically active effect on the region or organ to be treated.

Examples of bioactive agents include antineoplastic agents such as vincristine, vinblastine, vindesine, busulfan, chlorambucil, spiroplatin, cisplatin, carboplatin, methotrexate, adriamycin, mitomycin, bleomycin, cytosine arabinoside, arabinosyl adenine, mercaptopurine, mitotane, procarbazine, dactinomycin (antinomycin D), daunorubicin, doxorubicin hydrochloride, taxol, plicamycin, aminoglutethimide, estramustine, flutamide, leuprolide, megestrol acetate, tamoxifen, testolactone, trilostane, amsacrine (m-AMSA), asparaginase (Lasparaginase), etoposide, interferon a-2a and 2b, blood products such as hematoporphyrins or derivatives of the foregoing; biological response modifiers such as muramylpeptides; antifungal agents such as ketoconazole, nystatin, griseofulvin, flucytosine, miconazole or amphotericin B; hormones or hormone analogues such as growth hormone, melanocyte stimulating hormone, estradiol, beclomethasone dipropionate, betamethasone, cortisone acetate, dexamethasone, flunisolide, hydrocortisone, methylprednisolone, paramethasone acetate, prednisolone, prednisone, triamcinolone or fludrocortisone acetate; vitamins such as cyanocobalamin or retinoids; enzymes such as alkaline phosphatase or manganese superoxide dismutase; antiallergic agents such as amelexanox; anticoagulation agents such as warfarin, phenprocoumon or heparin; antithrombotic agents; circulatory drugs such as propranolol; metabolic potentiators such as glutathione; antituberculars such as p-aminosalicylic acid, isoniazid, capreomycin sulfate, cyclosexine, ethambutol, ethionamide, pyrazinamide, rifampin or streptomycin sulphate; antivirals such as acyclovir, amantadine, azidothymidine, ribavirin or vidarabine; blood vessel dilating agents such as diltiazem, nifedipine, verapamil, erythritol tetranitrate, isosorbide dinitrate, nitroglycerin or pentaerythritol tetranitrate; antibiotics such as dapsone, chloramphenicol, neomycin, cefaclor, cefadroxil, cephalexin, cephradine, erythromycin, clindamycin, lincomycin, amoxicillin, ampicillin, bacampicillin, carbenicillin, dicloxacillin, cyclacillin, picloxacillin, hetacillin, methicillin, nafcillin, penicillin or tetracycline; antiinflammatories such as diflunisal, ibuprofen, indomethacin, meclefenamate, mefenamic acid, naproxen, phenylbutazone, piroxicam, tolmetin, aspirin or salicylates; antiprotozoans such as chloroquine, metronidazole, quinine or meglumine antimonate; antirheumatics such as penicillamine; narcotics such as paregoric; opiates such as codeine, morphine or opium; cardiac glycosides such as deslaneside, digitoxin, digoxin, digitalin or digitalis; neuromuscular blockers such as atracurium mesylate, gallamine triethiodide, hexafluorenium bromide, metocurine iodide, pancuronium bromide, succinylcholine chloride, tubocurarine chloride or vecuronium bromide; sedatives such as amobarbital, amobarbital sodium, apropbarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, secobarbital sodium, talbutal, temazepam or triazolam; local anaesthetics such as bupivacaine, chloroprocaine, etidocaine, lidocaine, mepivacaine, procaine or tetracaine; general anaesthetics such as droperidol, etomidate, fentanyl citrate with droperidol, ketamine hydrochloride, methohexital sodium or thiopental and pharmaceutically acceptable salts (e.g. acid addition salts such as the hydrochloride or hydrobromide or base salts such\as sodium, calcium or magnesium salts) or derivatives (e.g. acetates) thereof; and radiochemicals, e.g. comprising alpha-, beta-, or gamma-emitters such as, for instance 177Lu, 90Y or 131I. Of particular importance are antithrombotic agents such as heparin and agents with heparin-like activity such as antithrombin III, dalteparin and enoxaparin; blood platelet aggregation inhibitors such as ticlopidine, aspirin, dipyridamole, iloprost and abciximab; and thrombolytic enzymes such as streptokinase and plasminogen activator; analgesic such as codeine, fentanyl, Hydrocone, acetaminophen, oxycodone; drug with action on central or peripheric nervous systems such as antiepileptic, antiparkinsonian, Psycholeptic or Psychoanaleptic drugs; hormones. Other examples of bioactive agent include antibodies such as Adalimumab, Avelumab, Durvalumab Infliximab, Atezolizumab, Nivolumab, Bevacizumab, Pembrolizumab, Ramucirumab, Trastuzumab Pertuzumab, Ipilimumab, Panitumumab, Natalizumab, Cetuximab, or fragment of antibodies. Other examples of bioactive agent include nanoformulated drug such as Poractant alfa, Doxorubicin HCl liposome injection, Liposomal amphotericin B lipid complex, Liposomal amphotericin B, Liposomal morphine sulphate, Liposomal cytarabine, Liposomal vincristine, Liposomal irinotecan, Liposomal verteporfin, Liposomal daunorubicin and cytarabine, Albumin-bound paclitaxel. Other examples of bioactive agent also include genetic material such as nucleic acids, RNA, and DNA of natural or synthetic origin, including recombinant RNA and DNA. DNA encoding certain proteins may be used in the treatment of many different types of diseases. For example, tumour necrosis factor or interleukin-2 may be provided to treat advanced cancers; thymidine kinase may be provided to treat ovarian cancer or brain tumors; interleukin-2 may be provided to treat neuroblastoma, malignant melanoma or kidney cancer; and interleukin-4 may be provided to treat cancer. Diagnostic agents are any compound, composition or particle which may allow imaging enhancement in connection with diagnostic techniques, including, magnetic resonance imaging, X-ray, in particular computed tomography, optical imaging, nuclear imaging or molecular imaging. Examples of suitable diagnostic agents are, for instance, magnetite nanoparticles, iodinated compounds, such as Iomeprol®, or paramagnetic ion complexes, such as hydrophobic gadolinium complexes.

According to this invention, the administration of a vasospasm inhibitor (VI) can enhance the therapeutic/diagnostic effect of said bioactive agent administered in combination with therapeutic treatment US-MV.

The enhancement of the therapeutic/diagnostic effect of the bioactive agent is endowed by the vasospasm inhibitor through different mechanisms, as mentioned above. For instance, the vasospasm inhibitor may enhance (1) the concentration of the bioactive agent (e.g therapeutic or contrast agents) in the vascular compartment of the target area, (2) its extravasation through blood vessels, (3) its intracellular delivery and/or (4) blood perfusion.

Said bioactive agent can be administered as separate formulation from the gas-filled microvesicles and/or included within the structure of the gas-filled microvesicles.

In the former case the bioactive agent is administered, for instance by injection, simultaneously or sequentially to the combined US-MV therapeutic treatment.

The bioactive agent can be a bioactive molecule (or mixture of bioactive molecules) or can be in the form of a suitable pharmaceutical composition comprising the bioactive molecule(s), such as a commercially available formulations on the market or in an extemporaneous preparation obtained by admixing the bioactive molecule with suitable aqueous carriers, which are preferably physiologically acceptable, comprise water (preferably sterile water), aqueous solutions such as saline (which may advantageously be balanced so that the final product for injection is not hypotonic), or solutions of one or more tonicity adjusting substances. Tonicity adjusting substances comprise salts or sugars, sugar alcohols, glycols or other non-ionic polyol materials (e.g. glucose, sucrose, sorbitol, mannitol, glycerol, polyethylene glycols, propylene glycols and the like), chitosan derivatives, such as carboxymethyl chitosan, trimethyl chitosan or gelifying compounds, such as carboxymethylcellulose, hydroxyethyl starch or dextran.

The bioactive agent is administered at an effective dose to the subject, wherein said effective dose is a dose suitable for exerting the therapeutic effect of the bioactive agent.

The bioactive agent can be administered either with a continuous infusion or by injecting at least one bolus of a certain volume of the suspension to the subject; more preferably said suspension of gas-filled microvesicles is administered as at least one bolus to the subject.

In an embodiment, the drug delivery protocol of the invention relates to a method for enhancing the efficacy of a combined therapeutic treatment of gas-filled microvesicles and ultrasound for therapy, said method comprising the steps of:

a′) administering a bioactive agent into the vascular system of a subject;

a) administering a vasospasm inhibitor into the vascular system of a subject;

b) administering a suspension of gas-filled microvesicles, into the vascular system of the subject;

c) applying ultrasounds for therapy to a region of interest of the subject.

In an embodiment of the present invention, step a′) and step a) of the disclosed method are carried out simultaneously or sequentially.

In an embodiment, step a′) and step a) are carried out simultaneously.

In another embodiment, step a′) and step a) are carried out sequentially.

In principle, steps a), a′) and/or b) can be carried out either simultaneously or sequentially, in any order, depending on the specific treatment and protocol, including combinations of simultaneous (of only two of the above steps) and sequential protocols of administrations.

For instance, steps a) and/or a′) can be carried out simultaneously or sequentially with respect to step b).

In an embodiment, step a) and/or step a′) are carried out simultaneously with step b).

In an alternative embodiment, steps a) and/or step a′) are carried out sequentially to step b). For instance, steps a)/a′) are carried out (simultaneously or sequentially to each other) from 1 second to 15 minutes before the step b), more preferably from 5 seconds to 12 minutes before, still more preferably at least 10 minutes before.

In a further embodiment, step a′) and b) can be carried out simultaneously. For instance, the suspension of microvesicles may contain the bioactive agent; said suspension can be administered simultaneously with the VI or sequentially, e.g. after administration of the VI.

In a further embodiment, the bioactive agent can be included within the structure of the gas-filled microvesicles through different mechanisms: i) it can be bound to an amphiphilic molecule of the gas-filled microvesicles through a covalent bond; ii) it may also be suitably associated to the gas-filled microvesicles via physical and/or electrostatic interaction, and iii) it can be a compound which is admixed with the components forming the gas-filled microvesicles to be eventually incorporated in the gas-filled microvesicles structure

In a further embodiment, the drug delivery protocol of the invention relates to a method for enhancing the efficacy of a combined therapeutic treatment of gas-filled microvesicles and ultrasound for therapy, said method comprising the steps of:

a) administering a vasospasm inhibitor into the vascular system of a subject;

b) administering a suspension of gas-filled microvesicles, comprising a bioactive agent, said bioactive agent being included within the structure of said gas-filled microvesicles, into the vascular system of the subject;

c) applying ultrasounds for therapy to a region of interest of the subject.

In an embodiment of the present invention, step a) and step b) of the disclosed method are carried out simultaneously or sequentially.

In an embodiment, step a) is carried out simultaneously with step b).

In an alternative embodiment, step a) is carried out sequentially to step b). Preferably, step a) is carried out from 1 second to 15 minutes before the step b), more preferably from 5 seconds to 12 minutes before, still more preferably at least 10 minutes before.

The suspension of gas-filled microvesicles comprising a bioactive agent can be administered either with a continuous infusion or by injecting at least one bolus of a certain volume of the suspension to the subject; more preferably said suspension of gas-filled microvesicles is administered as at least one bolus to the subject.

The suspension of gas filled microvesicles comprising a bioactive agent is administered at an effective dose to the subject, wherein said effective dose is such that both the gas filled microvesicles and the bioactive agent included in their structure are administered at a dose suitable for exerting their therapeutic effect.

The following examples will help to further illustrate the invention.

### EXAMPLES

Materials and Methods

Preparation of Formulation P01

Formulation P01 was prepared according to the procedure described in Ref. 17 (see Example 2). The amphiphilic materials in the formulation were DSPC:DPPE-PEG5000 in a respective molar ratio of 9:1. The concentration of MV in the solution was evaluated by Coulter Counter method (e.g. Coulter Counter Multisizer 3, equipped with the Multisizer 3 software).

Preparation of the Formulation P02

The procedure illustrated in the working examples of Ref. 16 was used for preparing the formulation (P02).

Briefly, an emulsion of cyclooctane and water (about 1.5/100 v/v) containing about 90 mg/L of DSPC, 7 mg/L of palmitic acid, 60 mg/L of DPPE-PEG5000 and 100 g/L of PEG4000 was prepared (Megatron MT3000, Kinematica; 10'000 rpm) and sampled into DINER vials (about 1 mL/vial).

The vials were cooled at −50° C. under vacuum and then subjected to lyophilization, followed by secondary drying above room temperature until complete removal of water and solvent (less than 0.5% by weight). At the end of the freeze-drying process, the headspace of the vials was saturated with a 35/65 mixture of C4F10/N2 and the vials were stoppered and sealed. The microvesicles solution was obtained by reconstitution of the lyophilized preparation (at concentrations of 15 mg/mL) in NaCl solution (0.9%) upon gentle agitation in the presence of a gas. The volume of reconstruction is adapted to obtain an adequate volume of injection for animal body weight.

Preparation of Definity®/Luminity®

The microvesicles were activated in accordance with the manufacturer recommendations. The concentration of microvesicles was defined by the Coulter counter method (e.g. Coulter Counter Multisizer 3, equipped with the Multisizer 3 software).

Administration

The volume of microvesicles suspensions is diluted if necessary, to obtain a bolus comprise in 200-500 μL for the rat, 50-200 μL for mice. Each injection is followed by a saline flush.

Boluses were spaced by 5 min, when the treatment duration was 20 min.

The dose of microvesicles is expressed in total number of microvesicles per kg, according to the protocol, model and species. The global dose could be split in several boluses, according to the protocol.

### Example 1

Enhancement of the Efficacy of a Combined Therapeutic Treatment of Gas-Filled Microvesicles and Ultrasound

Model and Animal Preparation

The model used was the NMU rat tumor model. Pre-pubescent rats (female, Sprague Dawley) received a single intra-peritoneal injection of 50 mg/kg of NMU (N-nitroso-N-methylurea, Sigma, Switzerland). Rats developing tumours were enrolled in protocol. In the NMU model, animals can develop several tumours allowing to have tumors receiving microvesicles and ultrasound for therapy; and tumors without ultrasound insonation (called untreated) in the same animal. The rats were anesthetized and placed on dorsal decubitus. A catheter was placed in the tail vein to inject drugs and microvesicles. The US treatment transducer was placed at 5 cm of the tumor (spaced by a water tank). The perfusion was followed by contrast echography (C10-3v probe, EpiQ 7G, Philips), placed near the tumour with a 40-45 degrees angle.

To evaluate the efficiency of molecular extravasation, a 150 kDa-dextran labeled with Cy©5.5 fluorophore was injected just before therapeutic treatment with ultrasounds and microvesicles.

Five experimental conditions were tested:
i) a first group of rats (untreated) was not exposed to US-MV (basal extravasation of CY5.5h);
ii) a second group of tumors was administered only with MV;
iii) a third group was pre-treated with the vasospasm inhibitor and then administered with MV;
iv) a fourth group of rats was exposed to the US-MV treatment and
v) a fifth group was pre-treated with the vasospasm inhibitor and then exposed to US-MV treatment.

For the US+MV treatment, the US for therapy were applied with an acoustic pressure of 600 kPa, with a pulse scheme of 1 ms ON/10 s OFF for a duration of 20 minutes.

For the study, the two MV formulations, namely P02 (prepared according to Material and Methods) or Definity, were administered at a dose of 8 μl of gas/kg, divided in 4 boluses, spaced of 5 minutes each. In term of MV number, this equivalent of gas volume corresponded to 1.2×10⁸/kg for the P02 formulation and 5.2×10⁹/kg for Definity.

For the pretreatment with vasospasm inhibitor, Nimodipine (Sigma Aldrich, Switzerland) powder was first diluted in DMSO (40 mg/ml); the injected dose (1.4 mg/kg) was extemporary prepared in NaCl 0.9% and injected 10 min before the treatment.

Tumors were then harvested from sacrificed animals; the fluorescence was quantified on image acquired ex vivo (Fluobeam® 700). The fluorescence was quantified by ImageJ software.

Results

The results showed that the administration of Nimodipine was able to enhance the drug delivery efficacy of the combined therapeutic treatment US-MV. The pre-treatment with the VI before the combined use of MV-US allowed to increase the extravasation of the Cy®5.5 dye in the tumour tissue of almost two times compared with the extravasation obtained in the other conditions. In particular, the mean fluorescence signal in the first group (only CY5.5h) was found to be 1020 RFU/px/ms, in the second group (only MV) was 930 RFU/px/ms, in the third group (VI+MV) was 1030 RFU/px/ms and in the fourth group (US-MV) was 990 RFU/px/ms. Surprisingly, in the fifth group the mean fluorescence value, assessed after the pre-administration of VI followed by US-MV was found to be 2060 RFU/px/ms, i.e. a fluorescence value of twice with respect to the other groups.

### Example 2

Evaluation of Different US Parameters in Vasospasm Induction

Model and Animal Preparation

The model used for these experiments was the mesenteric rat microcirculation. Briefly, anesthetized animals were prepared to allow the visualization of an intestinal loop. The injections were made in the tail vein.

Rats were placed under the inverted microscope (Olympus® X2) equipped with a X20 objectives (mag final: 200). A numeric camera, driven by an open source software, acquired and recorded 30 s video at each time points required, in accordance with the protocol.

Preparation 01 was administered for this study.

US treatment was delivered by an experimental single element transducer (unfocused, plane, 1.5 MHz). Table 1 reports the tested US parameters, including the pulse scheme, the acoustic pressure and the duration of US application, for each protocol. The transducer was focus on the optical objective and placed to respect a working distance of 5 cm.

Image sequences were acquired, before, during and after treatment. Additional acquisitions could be performed every 5 min until 40 min after the initiation of US exposure to monitor the vasospasm resolution. A vasospasm is validated if a reduction of lumen of microvessels (i.e. venule, arteriole or capillaries) is observed, and/or a blood flow reduction and/or blood flow stop (compared to control conditions).

Results:

As reported in Table 1, results confirmed that a vasospasm occurred immediately after the first ultrasound pulse, at any tested acoustic pressure, for all the investigated treatment's duration (2, 5 and 20 min), microvesicle doses and administration modes (one or multiple boluses). The expression “ultrasound pulse” indicates a multicycle ultrasound wave transmitted by an ultrasound transducer and propagating within a medium. It is characterized by a duration called “Time ON” (e.g. 1 ms ON), a frequency, an amplitude.

TABLE 1
Experimental conditions and results.

| | | | US | Total MB | | Vasospasm |
|---|---|---|---|---|---|---|
| | Acoustic | | application | dose (number | Numbers | occurrence |
| | pressure | | Duration | microvesicles/ | of | (on 3 animals/ |
| MV | (kPa) | Pulse scheme | (Min) | kg | boluses | condition) |
| P01 | 200 | 1 msON/10 s OFF | 20 | 2.6E+08 | 4 | 3/3 100% |
| | 300 | 1 msON/10 s OFF | 20 | 2.6E+08 | 4 | 3/3 100% |
| | 400 | 1 msON/10 s OFF | 20 | 2.6E+08 | 4 | 3/3 100% |
| | 600 | 1 msON/10 s OFF | 20 | 2.6E+08 | 4 | 3/3 100% |
| | 400 | 1 msON/10 s OFF | 5 | 3.2E+08 | 1 | 4/4 100% |
| | 600 | 1 msON/10 s OFF | 5 | 3.2E+08 | 1 | 4/4 100% |
| | 800 | 1 msON/10 s OFF | 5 | 3.2E+08 | 1 | 4/4 100% |
| | 400 | 1 msON/10 s OFF | 2 | 3.2E+08 | 1 | 3/3 100% |
| | 600 | 1 msON/10 s OFF | 2 | 3.2E+08 | 1 | 3/3 100% |

### Example 3

Evaluation of Different Types of Pas-Filled Microvesicles in Vasospasm Induction

Model and Animal Preparation

The model used for these experiments was the mesenteric rat microcirculation. The model and animal preparation were described in example 2. This study aimed at the evaluation of different types of gas-filled microvesicles in the vasospasm induction. For this purpose, different microvesicles formulations were investigated, namely the formulation P02 and Luminity® (Lantheus) at different acoustic pressures (from 400 kPa up to 800 kPa). Table 2 reports the investigated experimental conditions.

### Results

As emerged from Table 2, results confirmed the occurrence of a vasospasm immediately after the first ultrasound pulse at any tested acoustic pressure and with any investigated formulation of gas-filled.

TABLE 2
Experimental conditions and results.

| | | | | Total MV | | Vasospasm |
|---|---|---|---|---|---|---|
| | Acoustic | | | dose (number | Numbers | occurrence |
| | pressure | | Duration | microvesicles/ | of | (on 3 animals/ |
| MV | (kPa) | Pulse scheme | (min) | kg | boluses | condition) |
| Luminity | 400 | 1 ms ON/10 sOFF | 5 | 3.2E+08 | 1 | 3/3 100% |
| Luminity | 600 | 1 ms ON/10 sOFF | 5 | 3.2E+08 | 1 | 3/3 100% |
| Luminity | 800 | 1 ms ON/10 sOFF | 5 | 3.2E+08 | 1 | 3/3 100% |
| P02 | 400 | 1 ms ON/10 sOFF | 5 | 3.2E+08 | 1 | 3/3 100% |
| P02 | 600 | 1 ms ON/10 sOFF | 5 | 3.2E+08 | 1 | 3/3 100% |
| P02 | 800 | 1 ms ON/10 sOFF | 5 | 3.2E+08 | 1 | 3/3 100% |

### Example 4

Study of Vasospasm Inhibition Mediated by a Calcium Channel Blocker in a Non-Tumor Model

Model and Animal Preparation

The model used for these experiments was the mesenteric rat microcirculation. The model and animal preparation were described in example 2.

This study aimed at the evaluation of the inhibition of vasospasm induced by a vasospasm inhibitor compound, in particular the dihydropiridine calcium channel blocker nimodipine.

For this purpose, the rats received a pre-treatment with nimodipine, 10 mins before the combined treatment of gas-filled microvesicles and ultrasound for therapy.

Nimodipine (Sigma Aldrich, Switzerland) powder was first diluted in DMSO (40 mg/ml); the injected dose (1.4 mg/kg) was extemporary prepared in NaCl 0.9% and injected 10 min before the treatment.

The treatment parameters were chosen in accordance with the previous studies to obtain the generation of a vasospasm. P01 formulation was administered for this study.

### Results:

Table 3 shows that the pre-treatment with VI was able to inhibit the occurrence of vasospasm in the 100% of the animals subjected to combined use of ultrasound for therapy and a suspension of gas-filled microvesicles.

TABLE 3
Experimental conditions and results.

| | | | | MV dose | | Vasospasm |
|---|---|---|---|---|---|---|
| | Acoustic | | | total (number | Numbers | occurrence |
| | pressure | | Duration | microvesicles/ | of | (on 3 animals/ |
| MV | (kPa) | Pulse scheme | (min) | kg | boluses | condition) |
| P01 | 400 | 1 ms ON/10 s OFF | 5 | 3.2E+08 | 1 | 0/3 0% |

### Example 5

Effects on the Tumour Perfusion of a US-Mediated Therapy Combined with Gas-Filled Microvesicles (DA3 Tumour Model).

Animal Model and Preparation

The mouse DA3 tumor model was used in these studies. The US treatment was delivered by the Verasonic® system (probe P4-2). The probe was placed at 5 cm from the tumor (spaced by a water tank, filled with degassed water).

Then, serial bolus injections of gas-filled microvesicles (P01) were performed and the tumor was submitted to therapeutic insonation (frequency, acoustic pressure and pulse characteristics as specified in the Table 4). The perfusion of each tumor was followed by the echographic signal of microvesicles (Verasonic® system). The signal was recorded and post-analysed with Vuebox® software. The occurrence of a vasospasm was demonstrated by the reduction of the perfusion (i.e. decrease of intensity of microvesicles signal and/or increase of replenishment time after US pulse, compared to control conditions).

The expression “replenishment time” indicates the time required to supply the area under the ultrasound beam with the vessel with fresh gas-filled microvesicles, after the substantial destruction of MV caused by each US pulse.

The expression “MV destruction” corresponds to the loss of integrity or structure of MV caused by acoustic activation of MV by US. This can be provoked for example by MV collapse or gas dissolution and monitored by acoustic signal follow up.

All parameters relative to microvesicles and ultrasound treatment were fixed (see Table 4). The therapeutic insonation parameters were chosen in accordance with the previous studies, to obtain a vascular permeabilization in the DA3 tumor. The P01 dose was chosen to allow a stable perfusion of tumor (1.3E+09 microvesicles/kg; divided in 4 boluses, injected every 6 min). Two groups of animals were tested: a group was pretreated with VI (Nimodipine, 1.4 mg/kg, iv, from a stock solution, 10 min before treatment) and compared with a group administered with only NaCl 0.9%).

Results

A decrease of perfusion between each US pulse was observed in the 100% of animals treated with saline. The tumour perfusion was stable in the 100% of the tested animals, after the pre-treatment with the VI, suggesting an inhibition of vasospasm. The results are summarized in Table 4.

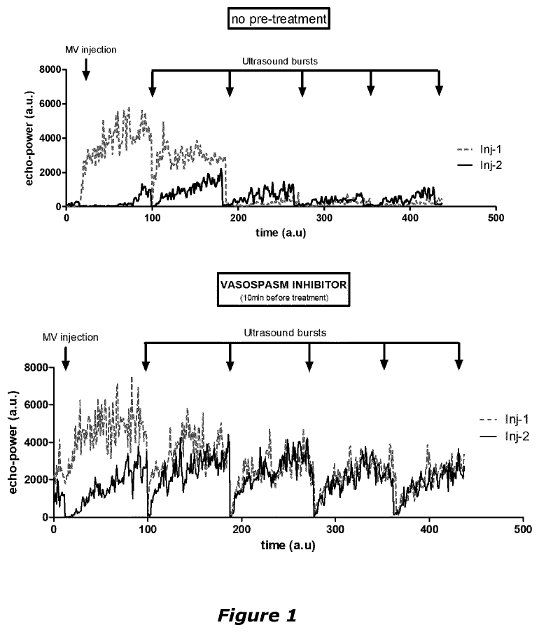

Moreover, FIG. 1 reports the quantification of tumoral perfusion during treatment with US+MV comparing the results obtained without VI pre-treatment (upper panel), with those obtained with a VI pre-treatment (Nimodipine 1.4 mg/kg IV) (lower panel). The results confirmed an improved tumour perfusion obtained after VI administration. In fact, the pre-treatment with VI was able to assure a more constant replenishment of microvesicles after the destruction by the US pulses than that obtained without any pre-treatment.

TABLE 4
Experimental conditions and results

| | | | | MV dose | | calcium | Decrease |
|---|---|---|---|---|---|---|---|
| | Acoustic | | | total (number | Number | channel | of |
| | pressure | | Duration | microvesicles/ | of | blockers | perfusion |
| MV | (kPa) | Pulse scheme | (min) | kg | boluses | treatment | in tumor |
| P01 | 800 | 1 ms ON/15 s OFF | 25 | 1.3E+09 | 4 | NaCl | 4/4 (100%) |
| | 800 | 1 ms ON/15 s OFF | 25 | 1.3E+09 | 4 | Nimodipine | 0/4 (0%) |
| | | | | | | (1.4 mg/kg) | |

### Example 6

Effects on the Tumour Perfusion of a US-Mediated Therapy Combined with Gas-Filled Microvesicles (NMU Tumour Model).

Model and Animal Preparation

Pre-pubescent rats (female, Sprague Dawley) received a single intra-peritoneal injection of 50 mg/kg of NMU (N-nitroso-N-methylurea, Sigma, Switzerland). The model is described in example 1.

Serial bolus injections of gas-filled microvesicles (as specified in Table 5) were performed and the tumor was submitted to therapeutic insonation (frequency, acoustic pressure and pulse characteristics as specified in the subsequent examples).

The perfusion of tumor during treatment was assessed as described in Example 5.

All parameters relative to microvesicles and ultrasound treatment were fixed (see Table 5). The therapeutic insonation parameters were chosen in accordance with the previous studies, to obtain a vascular permeabilization in the NMU tumor. The P02 dose was chosen to allow a stable perfusion of tumor (i.e. 1.2 E+09 microvesicles/kg, divided in 4 boluses; one bolus every 5 min).

Animals were pretreated with Nimodipine (1.4 mg/kg, iv, from a stock solution, 10 min before treatment, as detailed in example 3) and compared with the injection of NaCl 0.9%.

### Results

A decrease of contrast signal after the first US pulse was observed. All tumours presented a defect of perfusion after the first pulse.

Nevertheless, the rats pretreated with the dihydropiridine calcium channel blocker nimodipine (1.4 mg/kg, 10 min before) showed a stable tumor perfusion, across treatment, suggesting an inhibition of vasospasm.

TABLE 5
Experimental conditions and results.

| | | | | MV dose | | Calcium | Decrease |
|---|---|---|---|---|---|---|---|
| | Acoustic | | | total | Numbers | channel | of |
| | pressure | | Duration | (number | of | blockers | perfusion |
| MV | (kPa) | Pulse scheme | (min) | MV/kg | bolus | treatment | in tumor |
| P02 | 600 | 1 msON/15 s OFF | 20 min | 1.2E+09 | 4 | NaCl | 2/2 tumors |
| | | | | | | 0.9% | (100%) |
| P02 | 600 | 1 msON/15 s OFF | 20 min | 1.2E+09 | 4 | Nimodipine | 0/2 tumors |
| | | | | | | 1.4 mg/kg | (100%) |

### Example 7

Evaluation of Different Pharmacological Classes of Vasospasm Inhibitors in the Induction of a Vasospasm.

Model and Animal Preparation

The model used for these experiments was the mesenteric rat microcirculation. The model and animal preparation were described in example 2.

This study aimed at assessing the efficacy of different pharmacological classes of vasospasm inhibitor drugs currently used in clinic on the vasospasm inhibition.

All parameters relative to microvesicles and ultrasound treatment were fixed, as indicated in Table 6. The selected vasospasm inhibitor drugs and their associated dose are reported in Table 6. The duration between pre-treatment and injection of microvesicles and ultrasound treatment was in accordance with the pharmacocinetik pharmacokinetics and pharmacodynamics of each drug.
As reported in Table 6, results showed that dihydropiridine calcium blockers, such as nimodipine and nifedipine, the magnesium and nitrovasodilators, such as trinitrine, displayed a good ability to inhibit the vasospasm. The a-adrenoceptor antagonist Prazosin exhibited a lower ability, while the non-dihydropiridin calcium blocker Diltiazem and the ACE (Angiotensin-converting-enzyme) inhibitor Captopril did not show any relevant inhibiting activity toward the occurrence of the vasospasm.

TABLE 6
Experimental conditions and results.

| | | | | MV dose | | | | Vasospasm | |
|---|---|---|---|---|---|---|---|---|---|
| | Acoustic | | | total | Numbers | | Pre- | occurrence | |
| | pressure | | Duration | (number | of | | treatment | (on 3 animals/ | |
| MV | (kPa) | Pulse scheme | (min) | Mv/kg | bolus | VI tested | time | condition) | |
| P02 | 400 | 1 msON/10 s OFF | 5 | 1.5E+09 | 1 | Nimodipine | 10 min | 0/3 | 100% |
| | | | | | | 1.4 mg/kg | | | |
| P02 | 400 | 1 msON/10 s OFF | 5 | 1.5E+09 | 1 | Diltiazem | 10 min | 3/3 | 100% |
| | | | | | | 5 mg/kg | | | |
| P02 | 400 | 1 msON/10 s OFF | 5 | 1.5E+09 | 1 | Captoprill | 10 min | 3/3 | 100% |
| | | | | | | 25 mg/kg | | | |
| P02 | 400 | 1 msON/10 s OFF | 5 | 1.5E+09 | 1 | Nefedipine | 10 min | 0/3 | 0% |
| | | | | | | 0.5 mg/kg | | | |
| P02 | 400 | 1 msON/10 s OFF | 5 | 1.5E+09 | 1 | Mg²⁺ | 0 min | 0/3 | 0% |
| | | | | | | 300 mM | | | |
| P02 | 400 | 1 msON/10 s OFF | 5 | 1.5E+09 | 1 | Prazosin | 10 min | 1/3 | 33% |
| | | | | | | 0.5 mg/kg | | | |
| P02 | 400 | 1 msON/10 s OFF | 5 | 1.5E+09 | 1 | Trinitrine | 1 min | 0/3 | (0%) |

### Example 8

Additional Experiments Showing the Enhanced Efficacy of a Combined Therapeutic Treatment of Gas-Filled Microvesicles and Ultrasound

8.1 Disruption of Blood Clots

The model used is the healthy rats for which the middle cerebral artery of one of the brain hemispheres is occluded. This occlusion results in a decrease in blood perfusion in the correspondent hemisphere as characterized by the slop in the total vascular volume measured by Computed Tomography. To evaluate the efficiency of US-MV for blood clot disruption, the total vascular volume after US-MV treatment is compared to reference values (i.e. total vascular volume before and after MCA occlusion).

Six experimental conditions are tested:
vi) a first group of rats (untreated) is exposed to US (basal effect of US on clot lysis);
vii) a second group of rats is pre-treated with the vasospasm inhibitor and exposed to the US;
viii) a third group of rats is administered only with MV;
ix) a fourth group is pre-treated with the vasospasm inhibitor and then administered with MV;
x) a fifth group of rats is exposed to the US-MV treatment and
xi) a sixth group is pre-treated with the vasospasm inhibitor and then exposed to US-MV treatment.
The US therapy is applied on the occluded MCA with a frequency, about 0.25 to 2 MHz (e.g 1 MHz) and an acoustic pressure of about 0.4 to 1.5 MPa (e.g 0.6 MPa), with a pulse length about few ms up to few seconds ON (e.g 10 ms).

For the pretreatment with vasospasm inhibitor, the vasospasm inhibitor (e.g. Nimodipine, about 40 mg/kg) is administrated before the procedure, e.g. 5 minutes before administration of MV.

The MV formulations, is administered at a dose about 0.1 to 50 μl of gas/kg (e.g. 1 μl of gas/kg, 2·10⁸ MV/kg)). In term of MV number, this equivalent of gas volume corresponds to about 2×10⁷/kg up to 10×10⁹ MV/kg.

The administration of vasospasm inhibitor as illustrated above is able to enhance the efficacy of the reperfusion induced by the combined therapeutic treatment US-MV. Indeed, the pre-treatment with the VI before the combined use of MV-US allows a significant increase of the total vascular volume in comparison to the other conditions. Reperfusion can be observed to a lower extent in animals treated with only US+MV and to a much lower extent for animal treated with US or US+VI. Similar reperfusion is obtained for the group US and US+VI. No substantial effect on reperfusion is observed in the other groups: MV only or MV+VI.

8.2 BBB Opening

The model used are the healthy rat or brain tumour bearing rat. To evaluate the efficacy of US+MV on BBB opening, all animals receive an injection of a Blue Evan's Dye solution before receiving ultrasound treatment. The quantity of extravasated dye in the brain is correlated with the increased permeability of the blood brain barrier (BBB opening).

Six experimental conditions are tested:
i) a first group of rats (untreated) is exposed to US (basal effect of US on the BBBO);
ii) a second group of rats is pre-treated with the vasospasm inhibitor and exposed to the US;
iii) a third group of rats is administered only with MV;
iv) a fourth group is pre-treated with the vasospasm inhibitor and then administered with MV;
v) a fifth group of rats is exposed to the US-MV treatment and
vi) a sixth group is pre-treated with the vasospasm inhibitor and then exposed to US-MV treatment

For the pretreatment with vasospasm inhibitor, the vasospasm inhibitor (e.g. Nimodipine, about 40 mg/kg) is administrated before the procedure, e.g. 5 minutes before administration of MV.

The MV formulation (about 0.1 to 50 μl of gas/kg; e.g. 1 μl of gas/kg) is administrated just before the US application. In term of MV number, this equivalent of gas volume corresponds to about 2×10⁷/kg up to 10×10⁹ MV/kg (e.g. 2·10⁸ MV/kg).

The US therapy is applied through the skull (about 0.2 MHz to 2 MHz, e.g. 0.25 MHz) with an acoustic pressure range about 150 kPa to 1.8 MPa (e.g. 400 kPa) and a pulse length about few μs to tens of ms ON (e.g. 10 ms ON).

In comparison to other conditions the administration of vasospasm inhibitor in combination with US+MV treatment increases the quantity of Evan's blue extravasated in brain parenchyma reflecting the higher opening of the Blood Brain Barrier. BBBO can be observed to a lower extent in animals treated with US+MV and no BBBO is observed in the other groups: US or MV only; US or MV combined with vasospasm inhibitor.

8.3 MV Enhanced Thermal Ablation

The model is the healthy rabbit. The efficacy of the thermal ablation is highlighted by the elevation of the temperature of the hindlimb muscle when exposed to the ultrasound and the observation of a thermal lesion within this tissue after dissection. The temperature rising during the procedure is followed using a thermocouple probe inserted in the sonicated areas of the muscle bundles.

Six experimental conditions are tested:
i) a first group of rabbits(untreated) is exposed to US (basal effect of US on thermal ablation);
ii) a second group of rabbits is pre-treated with the vasospasm inhibitor and exposed to the US
iii) a third group of rabbits is administered only with MV;
iv) a fourth group of rabbits is pre-treated with the vasospasm inhibitor and then administered with MV;
v) a fifth group of rabbits is exposed to the US-MV treatment and
vi) a sixth group is pre-treated with the vasospasm inhibitor and then exposed to US-MV treatment.

For the pretreatment with vasospasm inhibitor, the vasospasm inhibitor (e.g. Nimodipine about 40 mg/kg) is administrated before the procedure (e.g 5 minutes)

The MV formulation (about 0.1 to 50 μl of gas/kg, e.g. 5 μl of gas/kg) is administrated just before US application. In term of MV number, this equivalent of gas volume corresponds to about 2×10⁷/kg up to 10×10⁹ MV/kg (e.g 10×10⁸ MV/kg).

The US therapy is applied on the muscle tissue (about 0.25 MHz to 2 MHz, e.g. 0.5 MHz) with an acoustic pressure range about hundreds of kPa to few MPa (e.g. 2,7 MPa) and a pulse length about few ms up to few seconds ON (e.g 15 s).

A temperature elevation provoking a thermal lesion is obtained using US or US+VI or US+MV or US+MV+VI. No difference is observed between the two groups US and US+VI. However, in comparison to US or US+VI, the of use of US+MV allows a higher and faster temperature elevation and to decrease the level of energy needed to achieve such temperature elevation and to induce such thermal lesions. This observed effect is enhanced in comparison to all the other groups when vasospasm inhibitor is administered in combination with US+MV. No thermal lesion is observed in the other groups: MV only or MV combined with vasospasm inhibitor.

8.4 Neuromodulation

The animal model used is the rat. The modulation of neuronal function induced by US+MV is evaluated by measuring modifications of the electrical activity of neurons, located in the somatosensorial brain cortex. The method used is named stimulus-driven somatosensory evoked potentials (SSEPs). This method evaluates the brain neuronal response measuring the SSEP waveforms (amplitude and latency) elicited by an electrical stimulation of the forepaws. Here we compare changes in waveform induced by US+MV. Six experimental conditions are tested:
i) a first group of rats (untreated) is exposed to US to evaluate the effect of US on neuronal response;
ii) a second group of rats is pre-treated with the vasospasm inhibitor and exposed to the US;
iii) a third group of rats is administered only with MV;
iv) a fourth group is pre-treated with the vasospasm inhibitor and then administered with MV;
v) a fifth group of rats is exposed to the US-MV treatment and
vi) a sixth group of rats is pre-treated with the vasospasm inhibitor and then exposed to US-MV treatment.

For all experiments a baseline signal is recorded prior the procedure in order to evaluate changes in waveform induced by the stimulation.

For the pretreatment with vasospasm inhibitor, the vasospasm inhibitor (e.g. Nimodipine about 40 mg/kg) is administrated before the procedure (e.g. 5 minutes).

The MV formulation (about μl of gas/kg, e.g. 1 μl of gas/kg) is administrated just before US application. In term of MV number, this equivalent of gas volume corresponds to about 2×10⁷/kg up to 10×10⁹ MV/kg, e.g. 2·10⁸ MV/kg.

The US therapy is applied on the brain (about 0.25 MHz to 2 MHZ, e.g. 0.5 MHz) with an acoustic pressure range about hundreds of kPa to few MPa (e.g. 0.4 MPa) and a pulse length about few ms up to few seconds ON (e.g. 10 ms).

The stimulation of the somatosensorial cortex by US+MV induces a decrease in SEPPs signal amplitude and an increase in the latency in comparison to baseline signal. The administration of VI in combination with US+MV stimulation induces a higher decrease in SEPPs amplitude and a higher increase of signal latency in comparison to US+MV. The level of energy needed in order to induce such signal modification is also lower in the group treated with US+MV+VI in comparison to the other groups. No change in wave form is observed for the US or US+VI or MV or US+VI.

8.5 Non-Thermal Ablation

The model is the healthy rat or rat bearing brain tumors. The brain is exposed to US in order to ablate tissue. The efficacy of the procedure is evaluated detecting the presence of treatment induced lesion as highlighted by i) changes in tissue hemodynamics ii) an increase level of necrosis and apoptosis in targeted tissue and iii) a reduction of tumor growth over the weeks.

Six experimental conditions are tested:
i) a first group of rats (untreated) is exposed to US (basal effect of US on Non-thermal ablation);
ii) a second group of rats is pre-treated with the vasospasm inhibitor and exposed to the US;
iii) a third group of rats is administered only with MV;
iv) a fourth group of rats is pre-treated with the vasospasm inhibitor and then administered with MV;
v) a fifth group of rats is exposed to the US-MV treatment and
vi) a sixth group is pre-treated with the vasospasm inhibitor and then exposed to US-MV treatment.

For the pretreatment with vasospasm inhibitor, the vasospasm inhibitor (e.g. Nimodipine about 40 mg/kg) is administrated before the procedure (e.g. 5 minutes).

The MV formulation (about 0.1 to 50 μl of gas/kg, e.g. 1 μl of gas/kg) is administrated just before US application. In term of MV number, this equivalent of gas volume corresponds to about 2×10⁷ MV/kg up to 10×10⁹ MV/kg. (e.g. 2·10⁸ MV/kg).

The US therapy is applied on tissue (about 0.25 MHz to 2 MHz, e.g. 1 MHz) with an acoustic pressure range about hundreds of kPa to few MPa (e.g. 1 MPa) and a pulse length about few ms up to few seconds ON (e.g. 20 ms).

In comparison to all the other conditions the administration of vasospasm inhibitor in combination with US+MV allows a higher reduction in blood flow and a higher level of necrosis and apoptosis in the treated tissue. This translates into a higher reduction of tumor growth over the week in comparison to all the other groups. Similar effects are observed to a lower extent in animals treated with US+MV. No lesion is observed in the groups: US or US+VI or MV or MV+VI.

8.6 Radiosensitization

The model is a Rat bearing tumors. The aim is to treat tumors enhancing the effect of radiation therapy (RT) using US+MV. The efficacy of the procedure is evaluated detecting the presence of treatment induced lesion as highlighted by i) changes in tissue hemodynamics ii) an increase level of cell death and apoptosis in tissue exposed to US+RT and iii) a reduction of tumor growth and an increased survival over the weeks.

Ten experimental conditions are tested:
i) a first group of rats is exposed to RT to evaluate the effect of the treatment alone;
ii) a second group rats is exposed to RT and vasospasm inhibitor;
iii) a third group of rats is exposed to US;
iv) a fourth group is exposed to US and vasospasm inhibitor;
v) a fifth third group of rats is administered only with MV;
vi) a sixth group is pre-treated with the vasospasm inhibitor and then administered with MV;
vii) a seventh group of rats is exposed to the US-MV treatment;
viii) an eighth group is pre-treated with the vasospasm inhibitor and then exposed to US-MV treatment;
ix) a ninth group is exposed to US-MV and RT treatment;
x) a tenth group is pre-treated with the vasospasm inhibitor and then exposed to US-MV and RT treatment.

For the pretreatment with vasospasm inhibitor (e.g. Nimodipine about 40 mg/kg) is administrated before the procedure (e.g. 5 minutes).

The MV formulation (about 0.1 to 50 μl of gas/kg, e.g. 1 μl of gas/kg) is administrated just before US application. In term of MV number, this equivalent of gas volume corresponds to about 2×10⁷/kg up to 10×10⁹ MV/kg (e.g. 2·10⁸ MV/kg).

The US therapy is applied on the tumor (about 0.25 MHz to 2 MHz, e.g. 0.25 MHz) The US therapy is applied on the tissue with an acoustic pressure about hundreds of kPa to few MPa (e.g. 600 kPa), pulse length few ms up to few seconds (e.g. 10 ms).

The radiation therapy is applied on the same tissue after the US treatment. In comparison to the other conditions, the administration of vasospasm inhibitor prior to RT+US+MV allows a higher reduction in blood flow and a higher level of cell death and apoptosis in treated tissue. This translates into a reduced tumor growth and a higher survival rate in comparison to the other groups. At a lower level MV+US+RT or MV+US+/−VI or RT+/−VI also decrease the blood flow and induced cell death and apoptosis in tumor tissue and decrease in tumor growth. This effect is superior in the group treated with MV+US+VI in comparison to MV+US. No difference is observed between RT or RT+VI. No effect is observed in groups treated with MV or MV+VI or US or US+VI.

8.7 Hyperthermia

The model is the rabbit bearing a tumor. The aim is to treat the tumor by a fluorescent-cytotoxic drug and enhancing the drug delivery by US hyperthermia-induced method.

The hyperthermia is evaluated by the monitoring of temperature in the tumor by MR thermometry method, allowing a temperature map in the treated area. The efficacy of drug delivery is evaluated by the quantification of fluorescence accumulation in tumor, obtained by fluorometry.
Seven sets of experimental conditions are tested. All groups received the cytotoxic drug.
i) a first group received only the cytotoxic drugs (basal accumulation of fluorescent signal in the tumor);
ii) a second group of rabbits is treated with US+/−cytotoxic drug;
iii) a third group of rabbits is pre-treated with the vasospasm inhibitor and exposed to the US+/−cytotoxic drug;
iv) a fourth group of rabbits is administered with MV+/−cytotoxic drug;
v) a fifth group of rabbits is pre-treated with the vasospasm inhibitor and then administered with MV+/−cytotoxic drug;
vi) a sixth group of rabbits is exposed to the US-MV treatment+/−cytotoxic drug and
xvii) a seventh group is pre-treated with the vasospasm inhibitor and then exposed to US-MV treatment+/−cytotoxic drug.

For the pretreatment with vasospasm inhibitor, the vasospasm inhibitor (e.g. Nimodipine about 40 mg/kg) is administrated before the procedure (e.g. 5 minutes).

The MV formulation (about 0.1 to 50 μl of gas/kg, e.g) is administrated just before US application. In term of MV number, this equivalent of gas volume corresponds to about 2×10⁷/kg up to 10×10⁹ MV/kg. (e.g 2·10⁸ MV/kg)

The US therapy is applied on the tumor (about 0.25 MHz to 2 MHz, e.g. 0.5 MHz) with an acoustic pressure range about 200 kPa to 1 MPa (e.g. 500 kPa) and a pulse length about 1 ms up to 10 seconds ON (e.g. 10 ms).

No substantial increase of temperature is observed for the groups VI or MV nor MV+VI alone. Hyperthermia is obtained using US or US+VI or US+MV or US+MV+VI. The level of energy required to reach and maintain the hyperthermia is reduced in group MV+US and reduced to a higher extent in group US+MV+VI.

For all group for which hyperthermia is effective, an increase of fluorescent signal accumulation is measured. This effect is more pronounced in US+MV+VI compared to MV+US, reflecting a higher delivery of cytotoxic drug with the combined use of US-MV and VI.

### REFERENCES

[1] Y. Wang et al., “Clinical study of ultrasound and microbubbles for enhancing chemotherapeutic sensitivity of malignant tumors in digestive system,” Chinese J. Cancer Res., vol. 30, no. 5, pp. 553-563, 2018.
[2] L. Auboire et al., “Microbubbles combined with ultrasound therapy in ischemic stroke: A systematic review of in-vivo preclinical studies,” PLoS One, vol. 13, no. 2, February 2018.
[3] J. H. Hwang, Y. Zhou, C. Warren, A. A. Brayman, and L. A. Crum, “Targeted venous occlusion using pulsed high-intensity focused ultrasound,” IEEE Trans. Biomed. Eng., vol. 57, no. 1, pp. 37-40, January 2010.
[4] J. Tu, H. Zhang, J. Yu, C. Liufu, and Z. Chen, “Ultrasound-mediated microbubble destruction: A new method in cancer immunotherapy,” OncoTargets and Therapy, vol. 11. Dove Medical Press Ltd., pp. 5763-5775, 2018.
[5] J. Blackmore, S. Shrivastava, J. Sallet, C. R. Butler, and R. O. Cleveland, “Ultrasound Neuromodulation: A Review of Results, Mechanisms and Safety,” Ultrasound in Medicine and Biology, vol. 45, no. 7. Elsevier USA, pp. 1509-1536, 1 Jul. 2019.
[6] H. Deng et al., “Ultrasound-stimulated microbubbles enhance radiosensitization of nasopharyngeal carcinoma,” Cell. Physiol. Biochem., vol. 48, no. 4, pp. 1530-1542, August 2018.
[7] D. J. Ching et al, “Effect of microbubble contrast agent during high intensity focused ultrasound ablation on rabbit liver in vivo” Eur J Radiol.; 81(4):e519-23, 2012 April
[8] M. A. Santos, “Microbubble-assisted MRI-guided focused ultrasound for hyperthermia at reduced power levels”, International Journal Of Hyperthermia, Vol. 35, no. 1, 591-603, 2018.
[9] C. D. Arvanitis, N. Vykhodtseva, F. Jolesz, M. Livingstone, and N. McDannold, “Cavitation-enhanced nonthermal ablation in deep brain targets: Feasibility in a large animal model,” J. Neurosurg., vol. 124, no. 5, pp. 1450-1459, May 2016.
[10] S. B. Raymond, J. Skoch, K. Hynynen, and B. J. Bacskai, “Multiphoton imaging of ultrasound/Optison mediated cerebrovascular effects in vivo,” J. Cereb. Blood Flow Metab., vol. 27, no. 2, pp. 393-403, February 2007.
[11] EP1017425(B1)
[12] WO9953964
[13] U.S. Pat. No. 10,456,483 B2
[14] U.S. Pat. No. 5,558,092A
[15] WO9115244A2
[16] WO2004069284A2
[17] WO2019170606A1
[18] U.S. Pat. No. 5,711,933A
[19] U.S. Pat. No. 6,333,021B1
[20] U.S. Pat. No. 6,723,303B1
[21] WO9409829A1
[22] A. R. Rezai et al., “Noninvasive hippocampal blood-brain barrier opening in Alzheimer's disease with focused ultrasound.”
G. Dimcevski et al., “A human clinical trial using ultrasound and microbubbles to enhance gemcitabine treatment of inoperable pancreatic cancer,” J. Control. Release, vol. 243, pp. 172-181, December 2016.

1.-5. (canceled)

6. A method for enhancing the efficacy of a combined therapeutic treatment of gas-filled microvesicles and ultrasound for therapy, said method comprising the steps of:
a) administering a vasospasm inhibitor into the vascular system of a subject;
b) administering a suspension of gas-filled microvesicles into the vascular system of the subject;
c) applying ultrasounds for therapy to a region of interest of the subject.

7. The method according to claim 6, wherein said vasospasm inhibitor is selected from the group consisting of dihydropyridine calcium blockers, a-blockers and nitrovasodilators.

8. The method according to claim 7, wherein said vasospasm inhibitor is selected from the group consisting of nimodipine, nifedipine, magnesium, prazosin and nitroglycerin.

9. The method according to claim 6, wherein said suspension of gas-filled microvesicles is administered with a continuous infusion or by injecting at least one bolus.

10. The method according to claim 6, wherein step a) and step b) of said method are carried out sequentially.

11. The method according to claim 10, wherein step a) is carried out from 1 second to 15 minutes before the step b).

12. The method according to claim 6, wherein said ultrasounds for therapy have an acoustic pressure comprised between 100 and 900 kPa.

13. The method according to claim 6, wherein said ultrasounds for therapy have a pulse length comprised between 5 μs and 60 s.

14. The method according to claim 6, wherein said ultrasounds for therapy are applied for a time comprised between 1 second and 170 minutes.

15. The method according to claim 6, wherein said ultrasounds for therapy have a frequency comprised between 20 kHz and 70 MHz.

16. A method for enhancing the efficacy of a combined therapeutic treatment of gas-filled microvesicles and ultrasound for therapy, said method comprising the steps of:
a′) administering a bioactive agent into the vascular system of a subject;
a) administering a vasospasm inhibitor into the vascular system of a subject;
b) administering a suspension of gas-filled microvesicles, into the vascular system of the subject;
c) applying ultrasounds for therapy to a region of interest of the subject.

17. A method for enhancing the efficacy of a combined therapeutic treatment of gas-filled microvesicles and ultrasound for therapy, said method comprising the steps of:
a) administering a vasospasm inhibitor into the vascular system of a subject;
b) administering a suspension of gas-filled microvesicles, comprising a bioactive agent, said bioactive agent being included within the structure of said gas-filled microvesicles, into the vascular system of the subject;
c) applying ultrasounds for therapy to a region of interest of the subject.

18.-20. (canceled)

21. The method according to claim 6, wherein step a) and step b) of said method are carried out simultaneously.
